(19) United States
(12) Patent Application Publication
(10) Pub. No.: US 2011/0009753 A1
(43) Pub. Date: Jan. 13, 2011

(54) Respiration Rate Trending for Detecting Early Onset of Worsening Heart Failure

(76) Inventors: Yi Zhang, Plymouth, MN (US); Viktoria A. Averina, Roseville, MN (US); Julie Thompson, Circle Pines, MN (US)

Correspondence Address: HOLLINGSWORTH & FUNK, LLC;Suite 320, 8500 Normandale Lake Blvd., Minneapolis, MN, 55437, US

(21) Appl. No.: 12/787,777

(22) Filed: May 26, 2010

Related U.S. Application Data
(60) Provisional application No. 61/224,719, filed on Jul. 10, 2009.

(51) Int. Cl. A61B 5/0205; A61B 5/08

(52) U.S. Cl. 600/484; 600/529

(57) ABSTRACT

Patient respiration is sensed from which respiration measurements are made, including a median respiration rate (MedRR) and a maximum respiration rate (MaxRR). Determinations are made as to whether an abnormality exists in MedRR and in MaxRR. An output indicative of the patient's tachypnea status is generated in response to determining the abnormality in MedRR and MaxRR.

### RELATED APPLICATIONS

This application claims the benefit of Provisional Patent Application Ser. No. 61/224,719, filed on Jul. 10, 2009, to which priority is claimed pursuant to 35 U.S.C. §119(e), and is related to U.S. patent application Ser. Nos. 11/300,675 filed on Dec. 14, 2005, 61/011,912 filed on Jan. 22, 2008, Ser. No. 12/356,289 filed on Jan. 20, 2009, and 61/224,721 filed on Jul. 10, 2009, which are hereby incorporated herein by reference in their respective entirety.

### FIELD OF THE INVENTION

The present invention relates generally to methods and systems for assessing a patient's heart failure status and, more particularly for detecting early onset of worsening of a patient's heart failure condition.

### BACKGROUND OF THE INVENTION

The human body functions through a number of interdependent physiological systems controlled through various mechanical, electrical, and chemical processes. The metabolic state of the body is constantly changing. For example, as exercise level increases, the body consumes more oxygen and gives off more carbon dioxide. The cardiac and pulmonary systems maintain appropriate blood gas levels by making adjustments that bring more oxygen into the system and dispel more carbon dioxide. The cardiovascular system transports blood gases to and from the body tissues. The respiratory system, through the breathing mechanism, performs the function of exchanging these gases with the external environment. Together, the cardiac and respiratory systems form a larger anatomical and functional unit denoted the cardiopulmonary system.

Various disorders that affect the cardiovascular system may also impact respiration. For example, heart failure is an abnormality of cardiac function that causes cardiac output to fall below a level adequate to meet the metabolic demand of peripheral tissues. Heart failure (HF) is usually referred to as congestive heart failure due to the accompanying venous and pulmonary congestion. Heart failure may have a variety of underlying causes, including ischemic heart disease (coronary artery disease), hypertension (high blood pressure), and diabetes, among others.

Various types of disordered respiration are associated with HF. Respiration rate is linked to the patient's physical condition and is indicative of the patient's disease or health state. In some types of chronic diseases, changes in respiratory rate are gradual over time and may be measured over months or years. However, in heart failure decompensation, increases in respiratory rate can occur over hours or days. Clinical data collected in the ambulatory setting has demonstrated a statistically significant difference between respiration rate distributions from healthy subjects as compared to HF patients.

Rapid shallow breathing is one of the cardinal signs of heart failure. When the patient at rest spends more time at higher respiration rates, this is indicative of a worsening of their HF status. The appearance of rapid, shallow breathing in a HF patient is often secondary to increased pulmonary edema, and can indicate a worsening of patient status. An abnormally high respiration rate thus can be an indicator of HF decompensation.

Symptoms of dyspnea are among the primary reasons that reduce patients' quality of life and are a primary reason why many HF patients return to the hospital during a HF decompensation episode. It is estimated that nearly one million hospital admissions for acute decompensated heart failure occur in the United States each year, which is almost double the number admitted 15 years ago. The re-hospitalization rates during the 6 months following discharge are as much at 50%. Nearly 2% of all hospital admissions in the United States are for decompensated HF patients, and heart failure is the most frequent cause of hospitalization in patients older than 65 years. The average duration of hospitalization is about 6 days. Despite aggressive therapies, hospital admissions for HF continue to increase, reflecting the prevalence of this malady.

Because of the complex interactions between the cardiovascular, pulmonary, and other physiological systems, as well as the need for early detection of various diseases and disorders, an effective approach to monitoring and early diagnosis is needed. Accurately characterizing patient respiration aids in monitoring and diagnosing respiration-related diseases or disorders. Evaluating patient respiration information may allow an early intervention, preventing serious decompensation and hospitalization.

### SUMMARY OF THE INVENTION

The present invention is directed to systems and methods for implementing respiration rate trending methodologies for assessing a patient's heart failure status. In particular, the present invention is directed to systems and methods for implementing respiration rate trending methodologies for detecting early onset of worsening of a patient's heart failure. The present invention is directed to systems and methods for generating an alert in response to detecting early onset of worsening of a patient's heart failure.

Embodiments of the invention are directed to methods that involve sensing respiration and measuring a median respiration rate (MedRR) and a maximum respiration rate (MaxRR) using the sensed respiration. Method embodiments further involve determining whether an abnormality exists in MedRR and in MaxRR, and generating an output indicative of the patient's tachypnea status in response to determining the abnormality in MedRR and MaxRR.

For example, determining the abnormality in MedRR and MaxRR may involve computing a baseline (e.g., a long-term measure) for MedRR and MaxRR, respectively, computing a near-term measure (e.g., a current or short-term measure) for MedRR and MaxRR, respectively, and determining the abnormality in MedRR and MaxRR based on a comparison of a difference between the baseline and near-term measure relative to a predetermined threshold for MedRR and MaxRR, respectively. Method embodiments may also involve measuring a minimum respiration rate (MinRR) using the sensed respiration, determining whether there is an abnormal elevation in MinRR, and generating an output indicative of the patient's tachypnea status in response to determining the abnormality in MedRR and MaxRR and absence of abnormal elevation in MinRR.

According to various embodiments, medical systems may be implemented to include a respiration sensor configured to generate a signal indicative of patient respiration and respiration information circuitry coupled to the respiration sensor. The respiration information circuitry is configured to measure a median respiration rate (MedRR) and a maximum respiration rate (MaxRR) using the signal indicative of patient respiration. A processor is coupled to the respiration information circuitry and configured to determine whether an abnormality exists in MedRR and whether an abnormality exists in MaxRR.

An output device is coupled to the processor and configured to generate an output indicative of the patient's tachypnea status in response to determining the abnormality in MedRR and MaxRR. In some embodiments, the processor is configured to compute a baseline for MedRR and MaxRR, respectively, compute a near-term measure for MedRR and MaxRR, respectively, and determine the abnormality in MedRR and MaxRR based on a comparison of a difference between the baseline and near-term measure relative to a predetermined threshold for MedRR and MaxRR, respectively.

In further embodiments, the respiration information circuitry is configured to measure a minimum respiration rate (MinRR) using the signal indicative of patient respiration. The processor is configured to determine whether there is an abnormal elevation in MinRR, and the output device is configured to generate an output indicative of the patient's tachypnea status in response to determining the abnormality in MedRR and MaxRR and absence of abnormal elevation in MinRR.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a flow chart showing various processes of respiration rate trending in accordance with embodiments of the invention;

FIG. 2 is a flow chart that shows various processes for generating and using daily respiration metrics in accordance with embodiments of the invention;

FIGS. 3 and 4 illustrate an implementation for determining respiration metrics including daily maximum respiration rate, daily median respiration rate, and daily minimum respiration rate in accordance with embodiments of the invention;

FIG. 5 is a block diagram of a system for implementing various processes of respiration rate trending and alert generation in accordance with embodiments of the invention;

FIG. 6 is a flow chart illustrating various processes for detecting early onset of worsening of a patient's heart failure status in accordance with embodiments of the invention;

FIG. 7 graphically illustrates a significant improvement in detecting early onset of worsening heart failure status of patients when using both MaxRR and MedRR metrics in accordance with embodiments of the invention;

FIGS. 8-13 are flow charts illustrating various processes for detecting early onset of worsening of a patient's heart failure status in accordance with embodiments of the invention;

FIGS. 14A and 14B are pictorial representations of baseline and near-term respiration rate metrics and their respective thresholds for different levels of heart failure severity in accordance with embodiments of the invention;

FIGS. 15A-15C show different approaches to computing baseline and near-term averages for respiration rates in accordance with embodiments of the invention;

FIGS. 16A-16D are plots of different respiration rate metrics and activity data associated with methodologies for detecting early onset of worsening heart failure in accordance with embodiments of the present invention;

FIG. 17 is a flow chart illustrating various processes for detecting early onset of worsening of a patient's heart failure status in accordance with embodiments of the invention;

FIG. 18 illustrates a partial view of a patient implantable medical device that may be used to implement processes for detecting early onset of worsening of a patient's heart failure status in accordance with embodiments of the invention;

FIG. 19 is a block diagram of a medical system that may implement various diagnostic, alert, and/or therapy processes in accordance with various embodiments; and

FIG. 20 is a block diagram of one embodiment of a medical system that may be configured to implement respiration rate trending and alert processes in accordance with various embodiments of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof. The specification and drawings show, by way of illustration, various embodiments in which the invention may be practiced. It is to be understood that other embodiments may be utilized, and structural and functional changes may be made without departing from the scope of the present invention.

Systems, devices or methods according to the present invention may include one or more of the features, structures, methods, or combinations thereof described hereinbelow. For example, a device or system may be implemented to include one or more of the advantageous features and/or processes described below. It is intended that such device or system need not include all of the features described herein, but may be implemented to include selected features that provide for useful structures and/or functionality. Such a device or system may be implemented to provide a variety of therapeutic or diagnostic functions.

Physiological sensors used in conjunction with implantable devices provide opportunities for collection of patient data which may be analyzed to develop trends of patient status. These trends allow a physician to assess changes in patient health, to analyze the effects of therapy, and/or to track the progression and/or regression of a disease. Changes in respiration, for example, may be caused by various patient conditions. Causes of tachypnea (fast respiration rate), by way of example, may include various factors including exertion, fever, pain, anemia, obesity, pneumonia, pneumothorax, acute respiratory distress, heart failure, hyperthyroidism, abdominal distention, respiratory muscle paralysis, chronic obstructive pulmonary disease, and/or other conditions.

Information developed from respiration data in accordance with embodiments of the present invention provides for enhanced patient monitoring and therapy management, particularly when the status of a patient is in decline. In various embodiments of the invention, analysis of the patient's respiration, which may be used alone or in combination with other physiological information, provides for detection of early onset of worsening of the patient's heart failure status. In particular, early onset of worsening of the patient's heart failure status is detected by analysis of the patient's daily respiratory rate trend. Detection of early onset of worsening of the patient's heart failure status is enhanced by analysis of the patient's daily respiratory rate trend and activity level of the patient.

In various embodiments of the invention, analysis of the patient's respiration, which may be used alone or in combination with other physiological information, triggers an alert indicating a change in the patient status and/or effectiveness of therapy (e.g., pharmacological or cardiac stimulation therapy) delivered to the patient. In various embodiment, analysis of the patient's respiration alone or in combination with patient activity level triggers an alert indicating a deleterious change in the patient's tachypnea status. The processes described herein may be particularly effective in monitoring of patient status and therapy delivered to patients suffering from conditions such as heart failure. Some of the embodiments described herein are based on alert generation in conjunction with heart failure monitoring, although the invention is applicable to alert generation for any type of condition which causes a change in respiration, including the exemplary conditions producing tachypnea or other respiration pattern associated with worsening of heart failure.

Respiration rate has been shown to be predictive of mortality in a HF patient population. Dyspnea (primarily caused by tachypnea) is among the primary reasons for patients' reduced quality of life and are a primary reason why many HF patients return to the hospital during a HF decompensation episode.

In the chronic, non-decompensated state, heart failure patients have elevated respiration rates. These rates become even more highly elevated in association with decompensation even at rest. Thus, for many patients, respiration rate provides a valuable indication or prediction of impending acute decompensation of HF. Information developed from respiratory rate data in accordance with embodiments of the present invention provides for enhanced monitoring and therapy management of HF patients, particularly when the HF status of a patient is in decline. Particular embodiments of the invention provide for detection of early onset of worsening heart failure, which allows for early intervention and treatment.

Methodologies described herein advantageously provide physicians with a quantified respiration metric that can be used to monitor a patient's changing status and/or evaluate the effectiveness of therapy (e.g., drug or cardiac stimulation therapy) delivered to the patient. The methodologies used for developing respiration data involve measuring values of a respiration characteristic, which may be respiration rate, but could also be breath interval, tidal volume, and/or other respiration characteristics. The respiration characteristic measurements may be made for one or more breath cycles during a plurality of time apertures, which may or may not be overlapping in time. An estimated respiration characteristic, e.g., estimated rate, breath interval, tidal volume, etc, may be determined from the set of measured characteristic values for a particular aperture to summarize the measurements for the particular aperture. In one implementation, the median value of the respiration characteristic measurements made during an aperture is used to estimate the respiration characteristic of the aperture.

Other statistical estimates of respiration parameters (e.g., mean respiration rate) or non-statistical estimates (e.g., based on measured morphological characteristics of the respiration signal) may alternatively be used. The estimated respiration characteristics of a plurality of apertures may be used to develop a respiration trend, or may be used to derive a respiration metric that spans a period of time, such as a daily value. An estimated respiration characteristic may be estimated based on the measured respiration characteristic values of an individual aperture. Respiration metrics, such as daily respiration metrics, may be determined based on the estimated respiration characteristics of a plurality of apertures.

One implementation involves the use of a median estimator to determine daily respiration rate metrics, such as maximum respiration rate (MaxRR) and median respiration rate (MedRR) over a period of time. A daily minimum respiration rate (MinRR) may also be determined. Embodiments of the invention are directed to use of both MaxRR and MedRR to provide for enhanced monitoring of a patient's tachypnea status. In particular, it has been found through clinical investigation that the combined use of MaxRR and MedRR provides for superior detection of change in a patient's tachypnea status when compared to individual use of MaxRR or MedRR. It has been further found through clinical investigation that patient activity level can be used to reduce the occurrence rate of false positives. It has also been found through clinical investigation that the MinRR metric can be used to reduce the occurrence rate of false positives.

The daily maximum respiration rate may be best interpreted by considering it in association with the patient's daily activity. In a stable, active patient, the maximum respiration rate will be significantly higher than the minimum value, and will vary considerably from day to day, reflecting the variability in the patient's activities. If elevated maximum respiration rates are associated with periods of very limited activity, the patient may be experiencing exertional dyspnea even at low levels of exertion (for example, simply walking around the house, or climbing the stairs), which may indicate worsening patient status. A person whose activities are severely limited by health conditions may show less of a spread from minimum to maximum and/or less day-to-day variability in maximum respiration rate, due to limited, consistent daily activity patterns.

The median respiration rate is representative of the predominant respiration rate for a given time period. The daily median is relatively insensitive to transiently elevated respiration rates during periods of high activity, and also relatively insensitive to the lowest respiration rates typically occurring during deep sleep. The median corresponds closely to the resting respiration rate a physician observes during a clinic visit.

In various embodiments, a patient's daily maximum respiration rate and daily median respiration rate are determined. The patient's daily minimum respiration rate may optionally be determined. In these embodiments, the patient's respiration rate is measured for each breath cycle in a plurality of time apertures that cover about a 24 hour period. The median respiration rate is estimated for each time aperture. The daily maximum respiration rate is determined as the maximum median respiration rate of the time apertures spanning the 24 hour period. The daily median respiration rate may be determined as the median of the median respiration rates estimated for all of the time apertures that span the 24 hour period. In another implementation, the daily median respiration rate may be determined as the median value of all the respiration rate values measured over the 24 hour period. The daily minimum respiration rate is determined as the minimum median respiration rate of the time apertures spanning the 24 hour period.

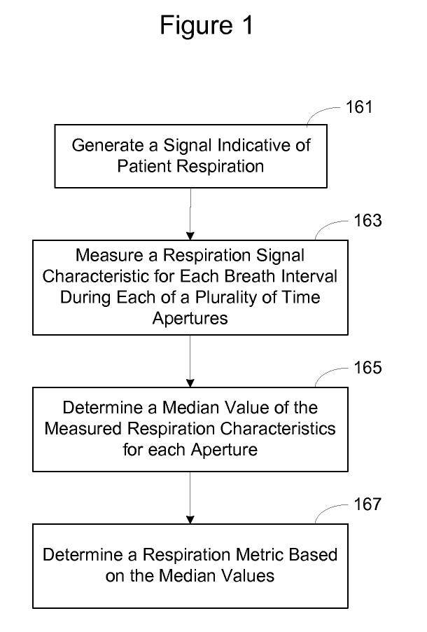

The use of median estimators to derive respiration metrics is illustrated in the flowchart of FIG. 1. Patient respiration is sensed and a signal indicative of patient respiration is generated 161. The patient respiration signal may be generated, for example, by any of a variety of implantable or patient external sensors, such as an implantable transthoracic impedance sensor, external respiratory bands having piezoelectric or other sensor elements, a respiratory mask flow sensor, or other types of respiration sensors. A characteristic of the respiration signal, such as respiration rate per breath cycle, is measured 163 during each of a plurality of time apertures. The median value of the respiration characteristic measurements for each aperture is determined 165 and is used to estimate the respiration characteristic for the aperture. For example, if respiration rate is the measured characteristic, the median value of the respiration rates measured for each breath cycle during the aperture is determined. The median value is used to estimate the respiration rate of the aperture. One or more respiration metrics are determined 167 based on the estimated respiration characteristics (e.g., median values) of the apertures.

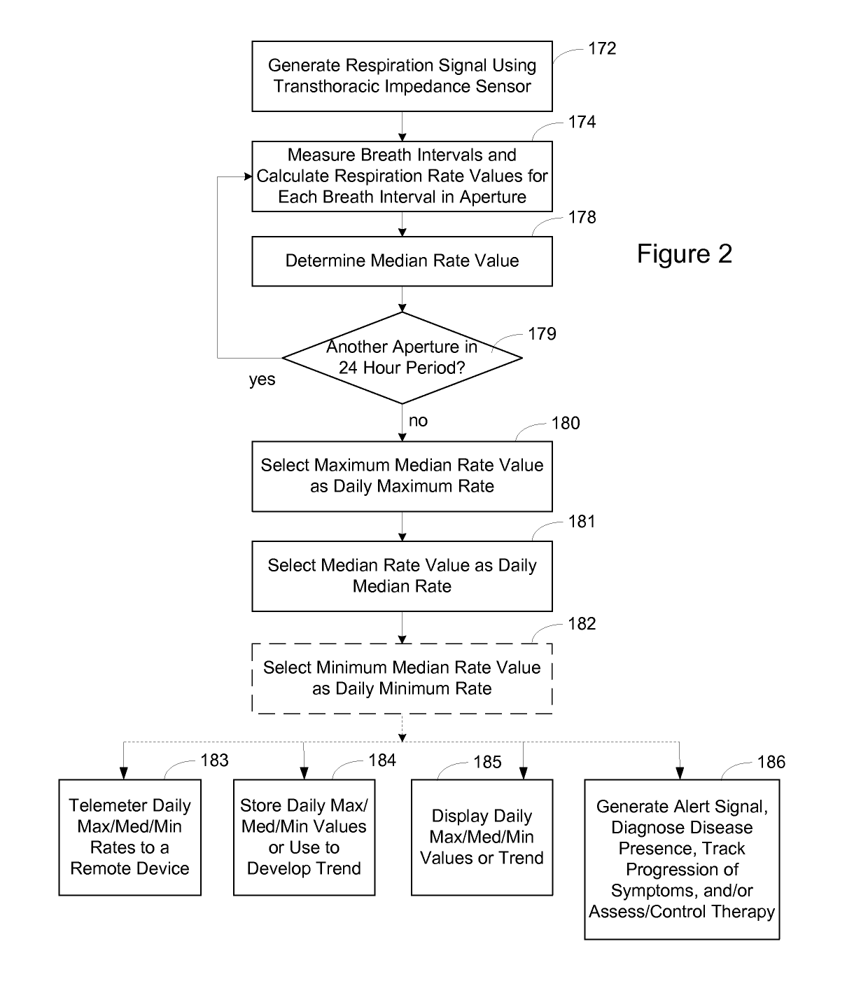

A method for generating and using daily respiration metrics is illustrated in FIG. 2. The process involves the use of an implantable transthoracic impedance sensor for determining a daily maximum and/or daily minimum respiration metric based on median estimators for the aperture respiration characteristics. In accordance with this embodiment, a respiration signal is generated 172 by a transthoracic impedance sensor implemented in conjunction with an implantable cardiac rhythm management (CRM) device or other implantable medical device. The transthoracic impedance sensor comprises intracardiac electrodes coupled to sensor drive/sense circuitry disposed within the CRM housing. The sensor drive circuitry delivers an electrical excitation signal, such as a strobed sequence of current pulses or other measurement stimuli, across the thorax via one set of the intracardiac electrodes.

In response to the drive current, a response voltage is sensed by the sense circuitry using another set of the intracardiac electrodes. The response voltage represents the transthoracic (i.e., across a portion of the chest or thorax) impedance. Transthoracic impedance sensing provides a voltage signal that tracks patient respiration and may be used to determine how fast and/or how deeply a patient is breathing. Additional aspects of transthoracic impedance sensing that may be utilized in conjunction with various embodiments of the present invention are described in commonly owned U.S. Pat. No. 6,076,015 which is incorporated herein by reference. In other embodiments, an external respiration sensor is used to detect patient respiration, it being understood that wholly external implementations of the present invention are contemplated.

A plurality of time apertures, covering about a 24 hour period, is superimposed relative to the generated respiration signal. The breath intervals occurring in each aperture are measured 174 and respiration rates for each breath cycle are calculated as the inverse of each measured breath interval. The median value of the measured respiration rates is computed and stored 178. Median values for each of the apertures are stored 179 throughout the 24 hour period. The maximum of the median values is selected 180 as the maximum daily respiration rate. The median of the median values is selected 181 as the median daily respiration rate. Optionally, the minimum of the median values is selected 182 as the minimum daily respiration rate.

The daily maximum and median rates (and optionally minimum rate) are stored or used 184 to develop trend data within the CRM device or remote device. The daily maximum and median rates (and optionally minimum rate) may optionally be telemetered 183 to a remote device. The daily maximum and median rates (and optionally minimum rate) or data developed from these metrics may optionally be displayed 185 on the device programmer screen or other user interface device as individual daily respiration metrics or as trended data.

The daily maximum and median rates (and optionally minimum rate) are preferably used 186 to generate an alert signal indicative of the patient's tachypnea status. The daily maximum and median rates (and optionally minimum rate) may be used 186 to generate an alert signal indicative of detection of early onset of worsening of the patient's heart failure status. The daily maximum and median rates (and optionally minimum rate) may be used 186 to generate an alert signal used for disease diagnosis, to track the progression of disease symptoms, and/or may to assess or control therapy. Although this example describes the use of daily metrics, other periodic metrics may also be determined, such as hourly metrics, weekly metrics, bi-weekly metrics, or monthly metrics. In addition, metrics other than maximum and minimum respiration rates may be determined, such as the daily, weekly, monthly, etc., median or mean respiration rates.

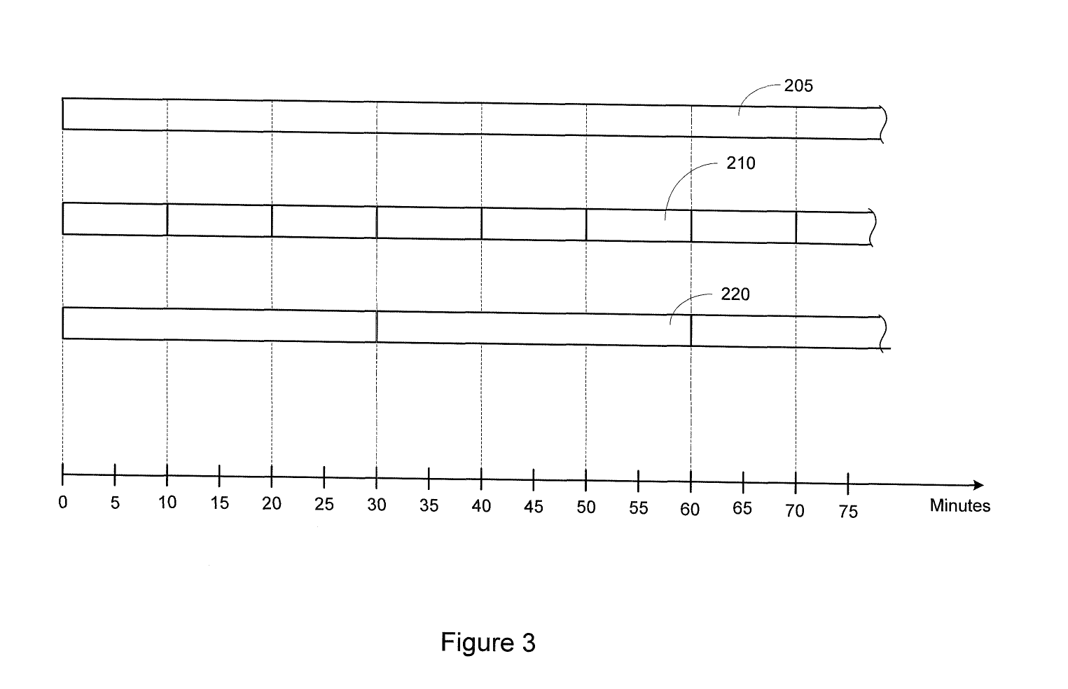

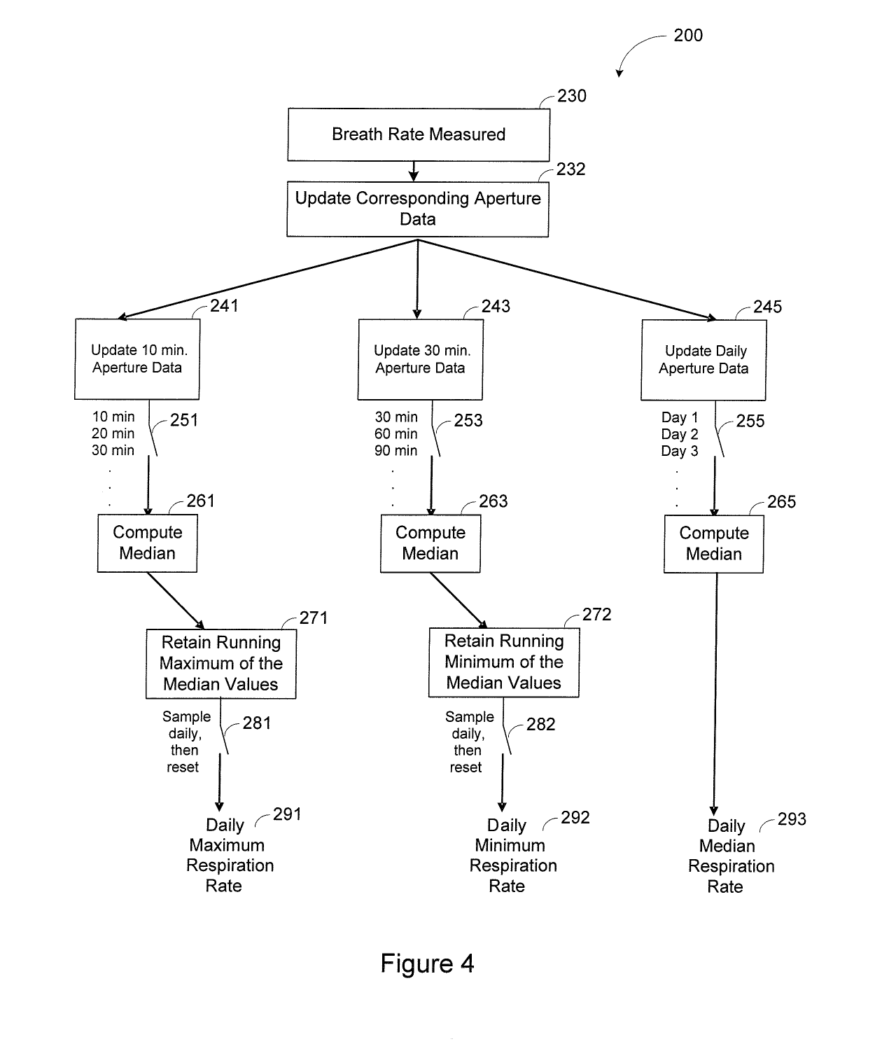

FIGS. 3 and 4 illustrate an implementation for determining respiration metrics including daily maximum respiration rate, daily median respiration rate, and daily minimum respiration rate in accordance with embodiments of the invention. Patient respiration is sensed and a respiration signal is generated. Overlapping apertures, as illustrated in FIG. 3, are superimposed on the respiration signal. The apertures include a 24 hour aperture 205 which is used to determine a daily median respiration rate. The apertures also include 10 minute apertures 210. The 10 minute apertures 210 are used to determine a daily maximum respiration rate. The apertures also include 30 minute apertures 220 which are used to determine a daily maximum respiration rate.

In one implementation, breath rates for each respiration cycle are measured and are used to determine median rates for an aperture. Several median rate processes are implemented, one corresponding to the median respiration rate of the 10 minute apertures, another corresponding to the median respiration rate of 30 minute apertures, and a third corresponding to a 24 hour median respiration rate. The daily minimum rate is determined from the median values of the 30 minute apertures 220 that span a 24 hour period. The daily maximum rate is determined from the median values of the 10 minute apertures 210 that span the 24 hour period. The daily median rate is the median value of the 24 hour period aperture 205. A process 200 for determining these daily metrics in accordance with one embodiment is illustrated in FIG. 4.

Breath rates are measured 230 from the respiration signal and used to acquire a daily minimum respiration rate, daily maximum respiration rate, and daily median respiration rate. The respiration signal may be generated, for example, by a transthoracic impedance sensor signal implemented in an implantable device, such as an implantable cardiac pacemaker or defibrillator. Breath detections received from the sensor may be pre-processed to avoid the use of spurious breath detections in determining the respiration metrics or trends. The process 200 may require that the breath rates meet certain criteria. In addition to providing breath rates for use in the respiration metric process 200, the respiration sensor circuitry, e.g., transthoracic impedance sensor, may provide data quality/status flags. Flags produced by the impedance sensor noise detection hardware/software may be used by the respiration metric process 200 to avoid using potentially corrupted data flagged as too noisy by the sensor. Further, the breath rates used to update the aperture data may be constrained to fall within a certain range of breath rates, e.g., about 4 breaths/minute to about 65 breaths/minute.

The measured respiration rate for the breath cycle is used to update 232 the data for each corresponding aperture. Data for each of the concurrently running apertures is updated 241, 243, 245 based on the measured breath rate. In some implementations, the breath rates may be computed in breaths/minute and the spacing of the histogram bins is 1 breath/minute. After an aperture is concluded, the median rate value for the aperture is computed 261, 263, 265. If an insufficient number of breaths are detected during an aperture, e.g., fewer than 100 breaths, then the aperture may be labeled invalid and a median for that aperture may not be computed. Throughout the 24 hour period, the running maximum of the median rate values for the 10 minute apertures is retained 271 and the running minimum of the median rate values for the 30 minute apertures is retained 272.

After the 24 hour period is concluded 281, 282, the daily maximum rate is reported 291, and the daily minimum rate is reported 292. The daily median respiration rate is determined 265 as the median rate value of the 24 hour aperture and reported 293. The daily maximum, minimum, and median values are preferably stored in the implantable medical device, and/or may be telemetered to a remote device, displayed on a display, or otherwise accessed by a physician or others. Additional information regarding respiration rate measurements which may be implemented in conjunction with the processes described herein is provided in commonly owned U.S. Patent Application Publication 2007/0135725 and the applications identified in the Related Application section of this disclosure, all of which are incorporated herein by reference.

Although various examples described herein provide an alert generated in response to a rise in respiratory rate above a threshold, those skilled in the art will appreciate that alerts may alternatively be generated upon respiration rate decreasing below a threshold. Aspects of the invention involve comparison of physiological parameters to alert criteria and generating an alert when the physiological parameters are equal to or beyond the alert criteria. Those skilled in the art will appreciate that a parameter value that is beyond a threshold can be, in various scenarios, either a parameter value below a threshold or a parameter value above a threshold.

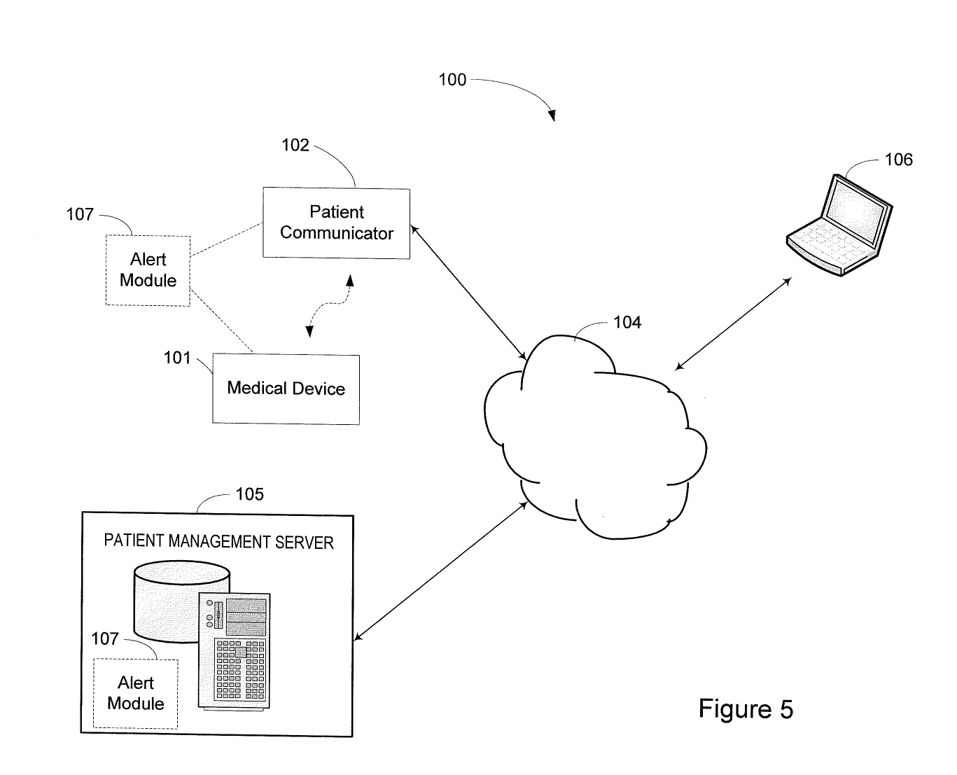

The diagram of FIG. 5 illustrates a system 100 that may be configured to implement the processes described herein. According to some embodiments, processes described herein may be implemented by all or a subset of the elements shown in FIG. 5. In some embodiments, a medical device 101 incorporates or otherwise is coupled to an alert module 107 that operate cooperatively to implement the processes described herein. In other embodiments, the medical device 101 incorporates or otherwise is coupled to an alert module 107 that operate cooperatively with a local or remote processing device or system (e.g., patient communicator 102, PC 106, and/or patient management server 105) to implement the processes described herein. It will be understood that various embodiments of the present invention can be implemented using all or selected elements (and other or alternative elements described herein) shown in FIG. 5. It will be further understood that some embodiments include at least one implantable element, while other embodiments include only external elements.

The following discussion of FIG. 5 presents an embodiment wherein information acquired by a medical device 101 and/or patient communicator 102 is transmitted to an alert module 107 of a patient management server 105. The alert module 107 is generally described as having the functionality to assess changes in patient status and/or therapy effectiveness based on comparison of parameters to alert criteria. It will be appreciated that the alert module 107 need not be located in the patient management server 105, but may alternatively be located in the medical device 101, the patient communicator 102, or the PC 106. It will be further appreciated that components of the alert module 107 may be incorporated across multiple devices 101, 102, 105, 106 (or other devices).

The patient is instrumented with an implanted, patient-worn, or patient-carried medical device 101 that communicates with a patient communicator 102. The patient communicator 102 may be a portable device, a bed-side device, a programmer, a PC 106 equipped with appropriate communication software and hardware, or other type of device configured to effect communications with the medical device 101 and the patient management server 105. For example, the medical device 101 may be a cardiac rhythm management (CRM) device or other type of implantable diagnostic and/or therapeutic device (e.g., respiration monitor) that is implanted in the patient. The medical device 101 and/or the patient communicator 102 are equipped with sensors configured to monitor various physiological parameters, including at least patient respiration. The medical device 101 stores information about the physiological parameters it senses and, at periodic intervals, on command, or on an event-driven basis, the medical device 101 downloads the stored physiological information to the communicator 102.

The patient communicator 102 is communicatively coupled to the patient management server 105 via a network 104, such as the Internet. The patent communicator 102 transmits the information acquired from the medical device 101 to the patient management server 105 for additional analysis. In addition to transmitting the information acquired by the medical device 101, the patient communicator 102 may also send to the patient management server 105 data that the patient communicator 102 has acquired through its own physiological sensors or other sensor with which the patient communicator 102 communicates, or via patient input.

At the patient management server 105, the data is stored and analysis of the patient condition and/or therapy effectiveness is performed by the alert module 107. As a part of this analysis, the physiological parameters are calculated and are compared to alert criteria. As previously mentioned, in alternate embodiments, computing and comparison of the respiration parameters to the alert criteria may be performed by the patient communicator 102 or by the implantable device 101. The parameter information may be trended and may be made available for remote access by a physician through a network-connected computer 106. When the parameters meet alert criteria, an alert signal may be generated to notify the physician or other action may be taken based on the alert signal.

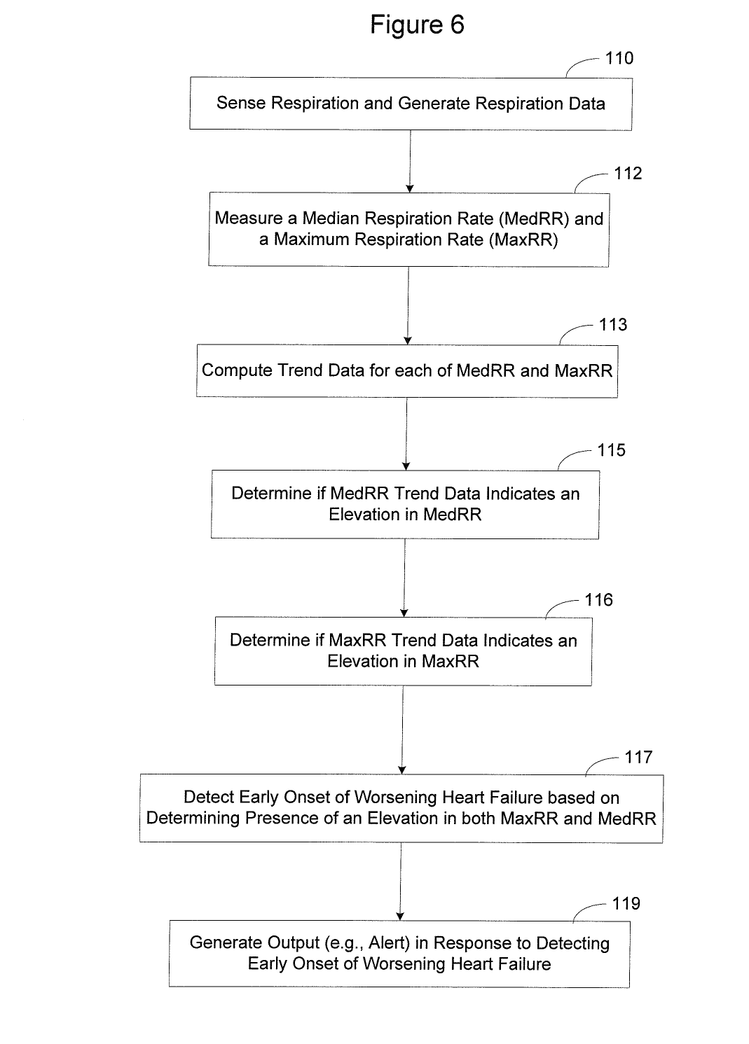

FIG. 6 is a flow chart illustrating various processes for detecting early onset of worsening of a patient's heart failure status in accordance with embodiments of the invention. The processes shown in FIG. 6 involve use of both maximum and median respiration rate metrics to provide for enhanced monitoring of a patient's tachypnea status. The combined use of MaxRR and MedRR has been found to enhance detection of a change in a patient's HF status when compared to detection techniques that only use MaxRR or MedRR. Although FIG. 6 illustrates an embodiment that uses both MaxRR and MedRR, in some embodiments, use of a single respiration rate metric, such as MedRR in particular, may adequately provide for detection of a change in a patient's HF status. Monitoring for changes in the patient's tachypnea status in accordance with embodiments of the present invention provides physicians the ability to detect early onset of worsening of the patient's heart failure status and provide early interventional therapies to prevent or moderate worsening of the patient's heart failure status.

According to the embodiment shown in FIG. 6, patient respiration is sensed 110 and respiration data is generated. A median respiration rate and a maximum respiration rate are measured 112. Trend data are computed 113 for each of the MedRR and MaxRR metrics. Processes are implemented to determine 115 if MedRR trend data indicates an elevation in MedRR and to determine 116 if MaxRR trend data indicates an elevation in MaxRR. Early onset of worsening heart failure is detected 117 based on determining presence of an elevation in both MaxRR and MedRR. An output is generated 119 in response to detecting early onset of worsening of the patient's heart failure status. Preferably, an alert is generated 119 indicating early onset of worsening of the patient's heart failure status, which is communicated to the patient's physician of health care advocate.

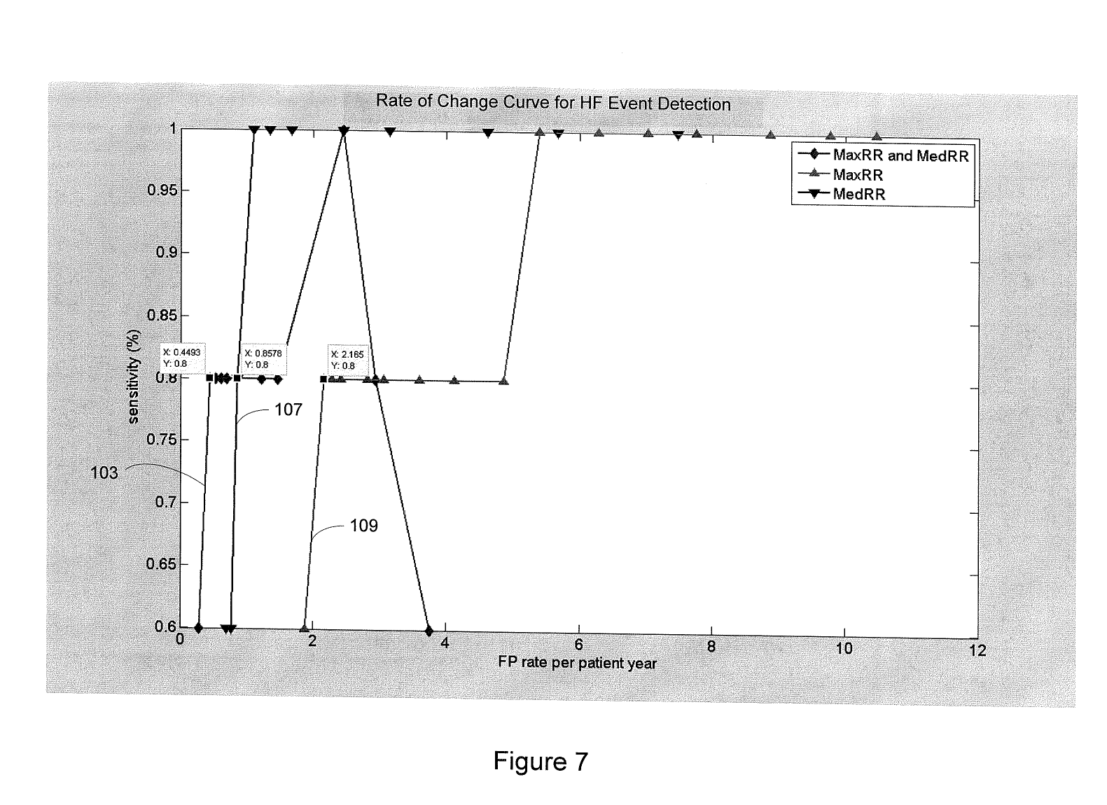

FIG. 7 graphically illustrates an improvement in detecting early onset of worsening heart failure status of patients when using both MaxRR and MedRR metrics in accordance with embodiments of the invention. FIG. 7 is a plot of the rate of false positives per patient year (x-axis) as a function of detection sensitivity (y-axis). Plots for MedRR 107, MaxRR 109, and combined MaxRR and MedRR 103 are shown in FIG. 7.

At an optimized detection sensitivity of 80%, the rates of false positives for the three HF event detection methodologies shown in FIG. 7 are as follows: HF event detection using MedRR 107 resulted in a false positive rate per patient year of approximately 0.86; HF event detection using MaxRR 109 resulted in a false positive rate per patient year of approximately 2.17; and HF event detection using combined MaxRR and MedRR 103 resulted in a false positive rate per patient year of approximately 0.45. It is noted that HF event detection using combined MaxRR and MedRR 103 at a detection sensitivity of 60% resulted in a false positive rate per patient year of approximately 0.29.

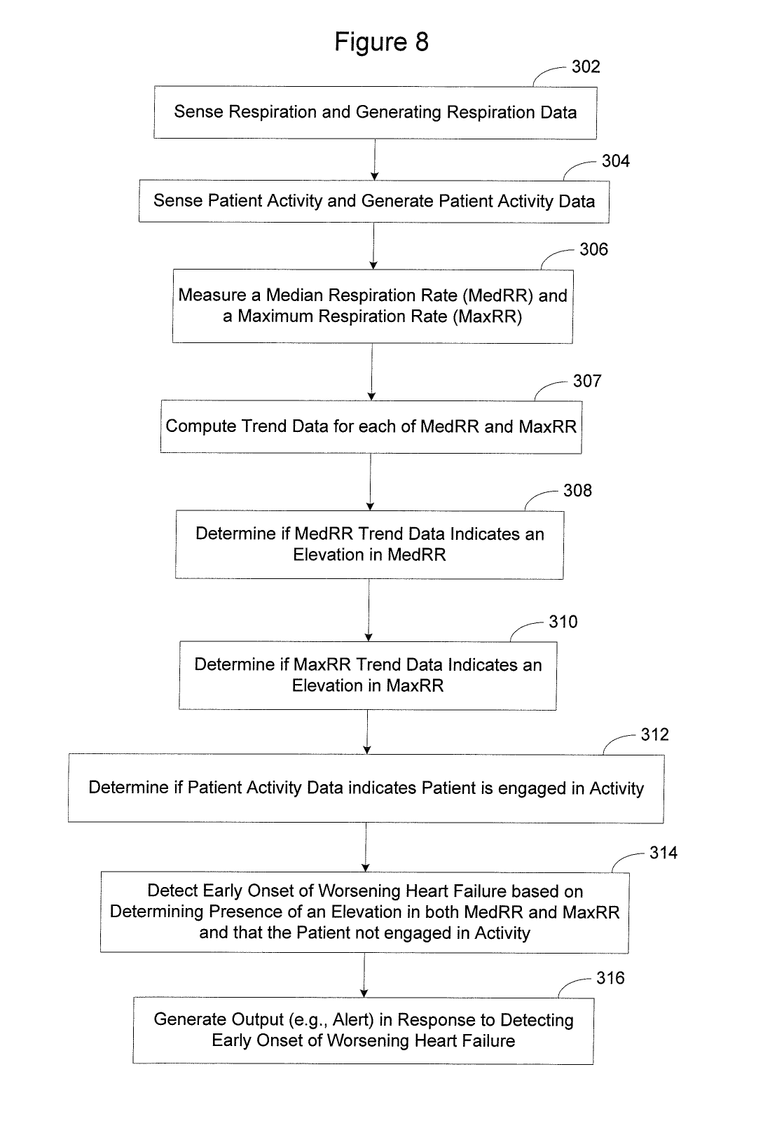

FIG. 8 is a flow chart illustrating various processes for detecting early onset of worsening of a patient's heart failure status in accordance with embodiments of the invention. According to the embodiment shown in FIG. 8, patient respiration is sensed 302 and respiration data is generated. Patient activity is sensed 304 from which patient activity data is generated. MedRR and MaxRR are measured 306. Trend data are computed 307 for each of MedRR and MaxRR. Processes are implemented to determine 308 if MedRR trend data indicates an elevation in MedRR and to determine 310 if MaxRR trend data indicates an elevation in MaxRR. Processes are implemented to determine 312 if the patient activity data indicates whether or not the patient is engaged in activity. Early onset of worsening heart failure is detected 314 based on determining presence of an elevation in both MedRR and MaxRR and that the patient is not engaged in activity (i.e., the detected elevation in RR is not due to patient activity). An output, such as an alert, is generated 316 in response to detecting early onset of worsening of the patient's heart failure status.

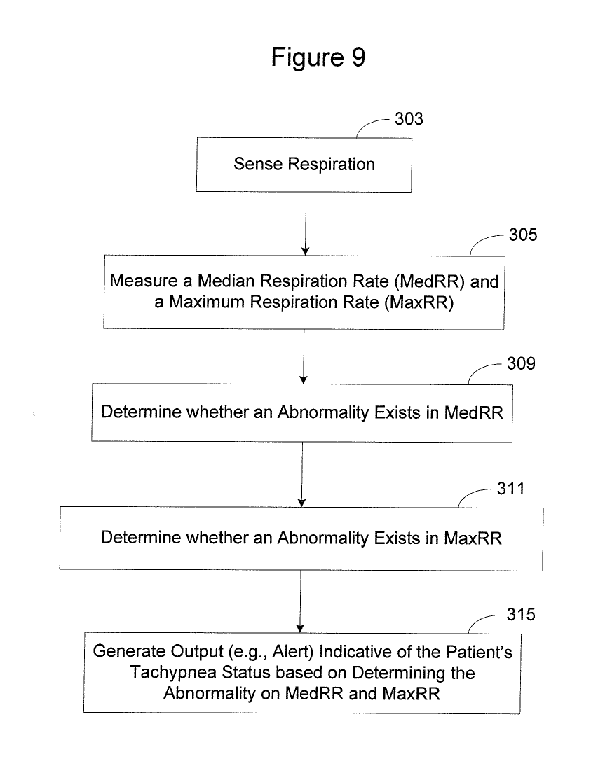

FIG. 9 is a flow chart illustrating various processes for detecting early onset of worsening of a patient's heart failure status in accordance with embodiments of the invention. According to the embodiment shown in FIG. 9, patient respiration is sensed 303 from which MedRR and MaxRR are measured 305. A determination is made 309 as to whether an abnormality exists in MedRR. A determination is made 311 as to whether an abnormality exists in MaxRR. An output, such as an alert, is generated 315 indicative of the patient's tachypnea status in response to determining the abnormality in MedRR and MaxRR.

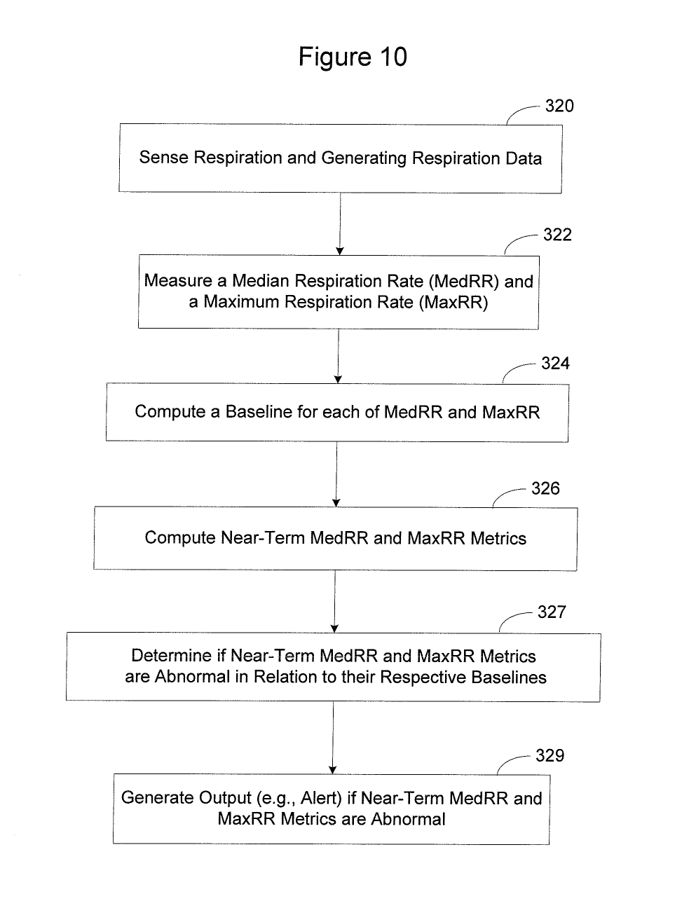

FIG. 10 is a flow chart illustrating various processes for detecting early onset of worsening of a patient's heart failure status in accordance with embodiments of the invention. According to the embodiment shown in FIG. 10, patient respiration is sensed 320 from which respiration data is generated. MedRR and MaxRR are measured 322. A baseline is computed 324 for each of MedRR and MaxRR. Metrics representative of the patient's near-term MedRR and MaxRR are respectively computed 326. Processes are implemented to determine 327 if the near-term MedRR and MaxRR metrics are abnormal in relation to their respective baselines. An output, such as an alert, is generated 329 if the near-term MedRR and MaxRR metrics are determined to be abnormal.

According to some embodiments, determining the abnormality in MedRR and MaxRR involves computing a baseline for MedRR and MaxRR, respectively, computing a near-term measure for MedRR and MaxRR, respectively, and determining the abnormality in MedRR and MaxRR based on a comparison of a difference between the baseline and near-term measure relative to a predetermined threshold for MedRR and MaxRR, respectively. The baseline may comprise one of a mean of a first predetermined period, a median of the first predetermined period, a weighted average of the first predetermined period, a predetermined value based on a population, and a predetermined value based on a patient's history. The near-term measure may comprise one of a mean of a second predetermined period and a median of the second predetermined period. The first predetermined period may longer than the second predetermined period or may overlap with at least a portion of the second predetermined period.

In various embodiments, determining the abnormality in MedRR and MaxRR may involve determining whether the difference between the baseline and near-term measure relative to their respective predetermined threshold exceeds their respective predetermined threshold for a predetermined duration of time. In other embodiments, determining the abnormality in MedRR and MaxRR may involve determining whether the difference between the baseline and near-term measure relative to their respective predetermined threshold exceeds their respective predetermined threshold for a predefined percentage of a predetermined duration of time. Determining the abnormality in MedRR and MaxRR may involve determining whether the respective abnormality in MedRR and MaxRR occur within a predetermined time window.

Determining the abnormality in MedRR and MaxRR may involve computing a variation of a baseline for MedRR and MaxRR, respectively, computing a variation of a near-term measure for MedRR and MaxRR, respectively, and comparing a difference between the baseline and near-term measure variation relative to a predetermined threshold for MedRR and MaxRR, respectively. Embodiments may involve one or more of these and other abnormality determinations, and may further involve adjusting at least one of the respective predetermined threshold, predetermined duration, predefined percentage, and predetermined time window based on desired performance requirements. Adjusting the respective predetermined threshold may be based on the respective baseline.

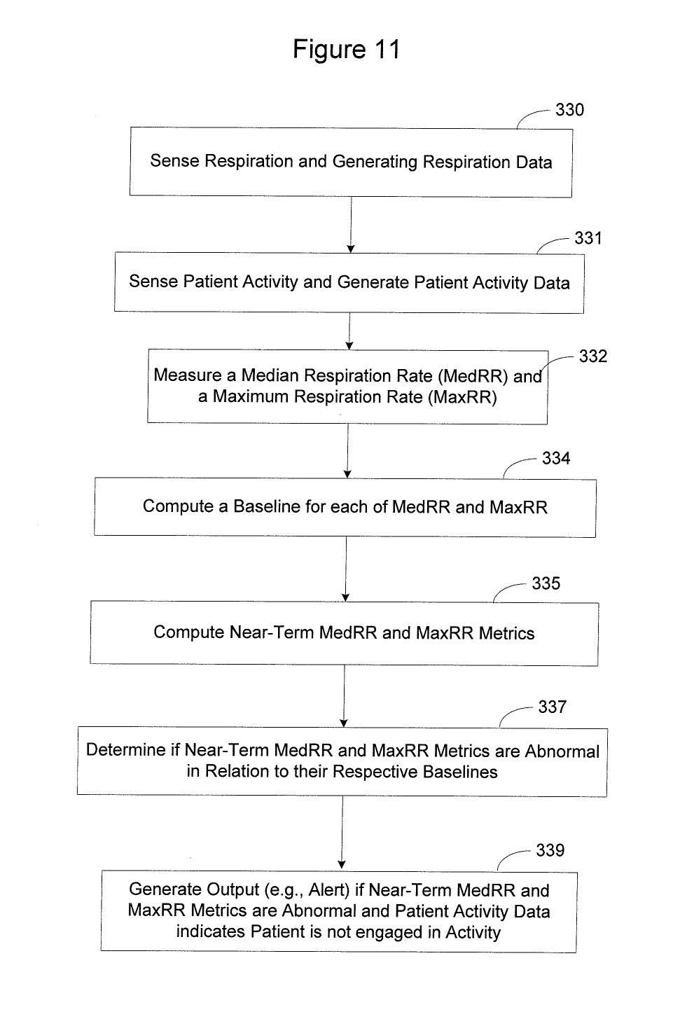

FIG. 11 is a flow chart illustrating various processes for detecting early onset of worsening of a patient's heart failure status in accordance with various embodiments of the invention. According to the embodiment shown in FIG. 11, patient respiration is sensed 330 from which respiration data is generated. Patient activity is sensed 331 from which patient activity data is generated. MedRR and MaxRR are measured 332. A baseline is computed 334 for each of MedRR and MaxRR. Near-term MedRR and MaxRR metrics are computed 335. Processes are implemented to determine 337 if the near-term MedRR and MaxRR metrics are abnormal in relation to their respective baselines. An output, such as an alert, is generated 339 if the near-term MedRR and MaxRR metrics are determined to be abnormal and the patient activity data indicates that the patient is not engaged in activity.

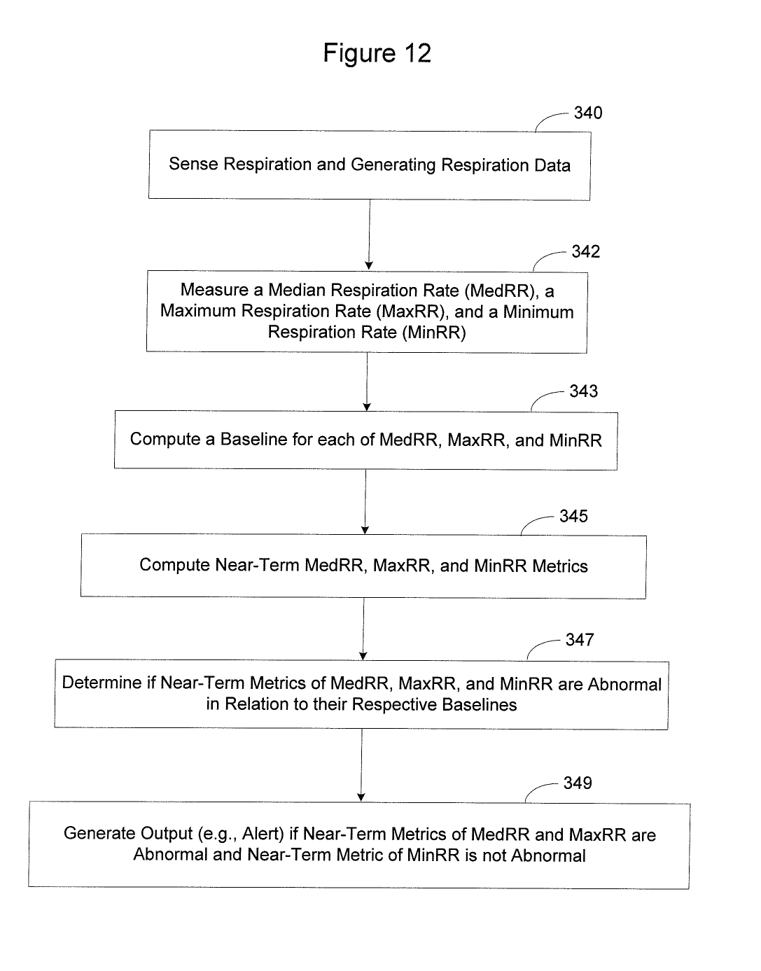

FIG. 12 is a flow chart illustrating various processes for detecting early onset of worsening of a patient's heart failure status in accordance with other embodiments of the invention. According to the embodiment shown in FIG. 12, patient respiration is sensed 340 from which respiration data is generated. MedRR, MaxRR, and MinRR are measured 342. A baseline is computed 343 for each of MedRR, MaxRR, and MinRR. Metrics representative of the patient's near-term MedRR, MaxRR, and MinRR are respectively computed 345. Processes are implemented to determine 347 if the near-term MedRR, MaxRR, and MinRR metrics are abnormal in relation to their respective baselines. An output, such as an alert, is generated 349 if the near-term MedRR and MaxRR metrics are determined to be abnormal and the near-term MinRR metric is determined not to be abnormal. It has been found that use of the MinRR metric in combination with the MedRR and MaxRR metrics provides for enhanced detection, as discussed previously.

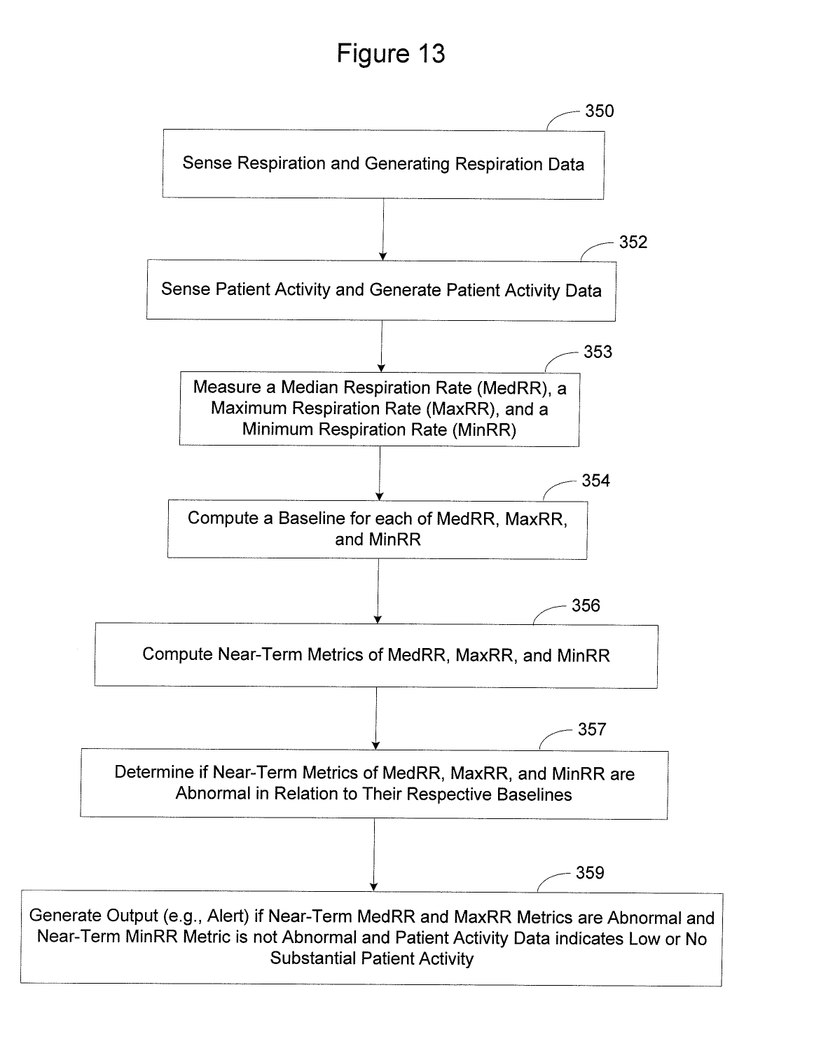

FIG. 13 is a flow chart illustrating various processes for detecting early onset of worsening of a patient's heart failure status in accordance with further embodiments of the invention. According to the embodiment shown in FIG. 13, patient respiration is sensed 350 from which respiration data is generated. Patient activity is sensed 352 from which patient activity data is generated. MedRR, MaxRR, and MinRR are measured 353. A baseline is computed 354 for each of MedRR, MaxRR, and MinRR. Metrics representative of the patient's near-term MedRR, MaxRR, and MinRR are respectively computed 356. Processes are implemented to determine 357 if the near-term MedRR, MaxRR, and MinRR metrics are abnormal in relation to their respective baselines. An output, such as an alert, is generated 359 if the near-term MedRR and MaxRR metrics are determined to be abnormal, the near-term MinRR metric is determined not to be abnormal, and the patient activity data indicates that the patient is not engaged in activity.

In accordance with further embodiments, the patient's heart failure status may be assessed at least in part by determining whether there is an abnormal relationship between any two of MedRR, MaxRR, and MinRR measurements. For example, if the algorithm determines that the difference between two of MedRR, MaxRR, and MinRR are outside of a predetermined normal range (i.e., a determination of how close these three metrics are), then the algorithm can generate an output indicating that the patient's heart failure status may be worsening. According to various embodiments, the patient's heart failure status may be assessed at least in part by determining whether a near-term measure of MinRR is greater than a baseline for MedRR. In other embodiments, the patient's heart failure status may be assessed at least in part by determining whether a near-term measure of MedRR is greater than a baseline for MaxRR. An output indicative of the patient's heart failure status may be generated in response to the heart failure status assessment and communicated to a processing device or system.

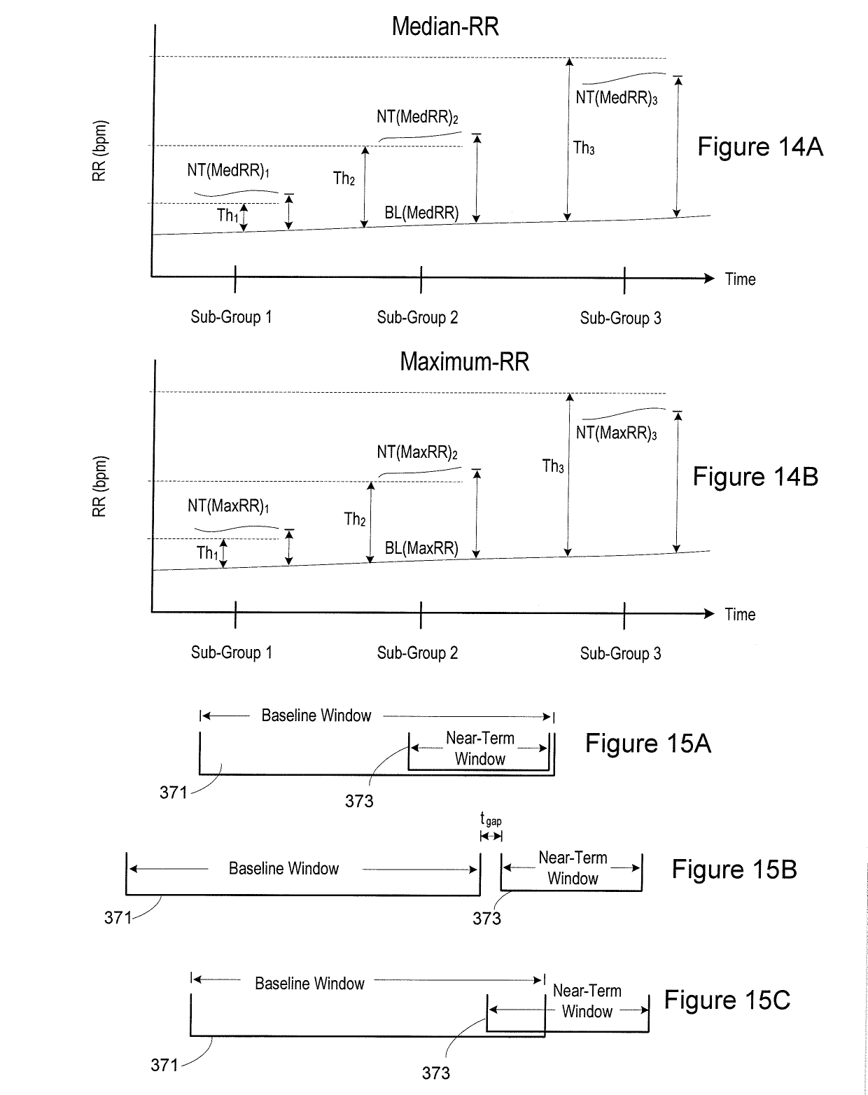

FIGS. 14A and 14B are pictorial representations of baseline and near-term respiration rate metrics and their respective thresholds for different sub-groups of patients, such as those having varying levels of heart failure severity (e.g., NYHA classes II, III, and IV) with different co-morbidities (e.g., pulmonary or renal co-morbidities). FIG. 14A shows a plot of baseline MedRR trend data in relation to plots of near-term MedRR trend data for a representative patient suffering from progressively worsening heart failure. Different thresholds, Th₁, Th₂, and Th₃ (shown in terms of respiration rate in breaths per minute) are defined to demarcate normal respiration from abnormal elevated respiration (e.g., tachypnea) for different classes of HF patients. FIG. 14B shows a plot of baseline MaxRR trend data in relation to plots of near-term MaxRR trend data for a representative patient suffering from progressively worsening heart failure. Different thresholds, Th₁, Th₂, and Th₃ (shown in terms of respiration rate in breaths per minute) are defined to demarcate normal respiration from abnormal elevated respiration for different classes of HF patients.

The thresholds shown in FIGS. 14A and 14B are preferably representative of a relationship between detection sensitivity and rate of false positives. Changes to a threshold modifies the relationship between detection sensitivity and the rate of false positives. The thresholds shown in FIGS. 14A and 14B may be established based on one or more factors, including patient population, NYHA classification, or comorbidities, for example. Thresholds may be the same or different for MedRR and MaxRR. Thresholds may be adjusted based on changes in patient condition, such as progression from HF class II to class III or presence/progression of one or more comorbidities.

Thresholds may be adjusted based on various factors, such as changes in baseline respiration rate metrics. A threshold may be adjusted as a function of change in a baseline respiration rate metric (e.g., a linear function, a step-wise function, etc.). Thresholds may be adjusted based on the mean of baseline respiration rate metric change over a specified time duration (e.g., a smaller threshold for a higher mean). Thresholds may be adjusted so that the algorithm operates at different sensitivity/specificity performance ranges or requirements. Adjusting a threshold of MinRR may provide a useful mechanism to triage a patient population into good, bad, and worse categories, for example, which could then be used to modify the MedRR and MaxRR change thresholds accordingly. Various other parameters of the algorithm may also be adjusted based on desired performance ranges or requirements, such as adjustments to one or more of the predetermined threshold, predetermined duration, predefined percentage, and predetermined time window described herein for determining whether an abnormality exists in MedRR, MaxRR, and MinRR metrics. Various thresholds and algorithm parameters may be adjusted, alone or in combination, to achieve different sensitivity/specificity performance ranges or requirements.

With reference to FIG. 14A, when the difference between NT(MedRR)₁ and BL(MedRR)₁ exceeds the threshold Th₁, this episode of patient breathing is considered abnormally elevated. If the difference between NT(MedRR)₃ and BL(MedRR)₃ fails to exceed the threshold Th₃, as is the case in FIG. 14A, this episode of patient breathing is considered elevated but normal. With reference to FIG. 14B, when the difference between NT(MaxRR)₁ and BL(MaxRR)₁ exceeds the threshold Th₁, this episode of patient breathing is considered abnormally elevated. If the difference between NT(MaxRR)₃ and BL(MaxRR)₃ fails to exceed the threshold Th₃, as is the case in FIG. 14B, this episode of patient breathing is considered elevated but normal.

FIGS. 15A-15C show different approaches to computing baseline and near-term averages for respiration rates. A baseline window 371 preferably has a length, typically in terms of number of days, so that a sufficiently high percentage of valid data can be acquired for computing a meaningful baseline average respiration rate metric. A useful length for a representative baseline window 371 may be on the order of 40 days. Various known methods for computing a baseline average for a respiration metric may be used, such as a moving average function, a median function or a running average, for example. A near-term window 373 preferably has a length, typically in terms of number of days, so that a snapshot of the patient's current (e.g. instantaneous) or near-term respiration rate data can be obtained for computing a meaningful near-term average respiration rate metric. A useful length for a representative near-term window 373 may be on the order of a few days, such as 3 days.

In FIG. 15A, a near-term window 373 is defined within a baseline window 371. In FIG. 15B, the near-term window 373 is defined outside of the baseline window 371 and separated by a gap, t_{gap}. The gap and length of the near-term window 373 are defined so that no significant change in the baseline respiration rate metric computed using the baseline window 371 is seen during the combined duration of the gap and near-term window 373. An advantage to the windowing scenario depicted in FIG. 15B is that the baseline data are not tainting the near-term data. In another embodiment, there is an overlap in the two windows 371 and 373, as is shown in FIG. 15C.

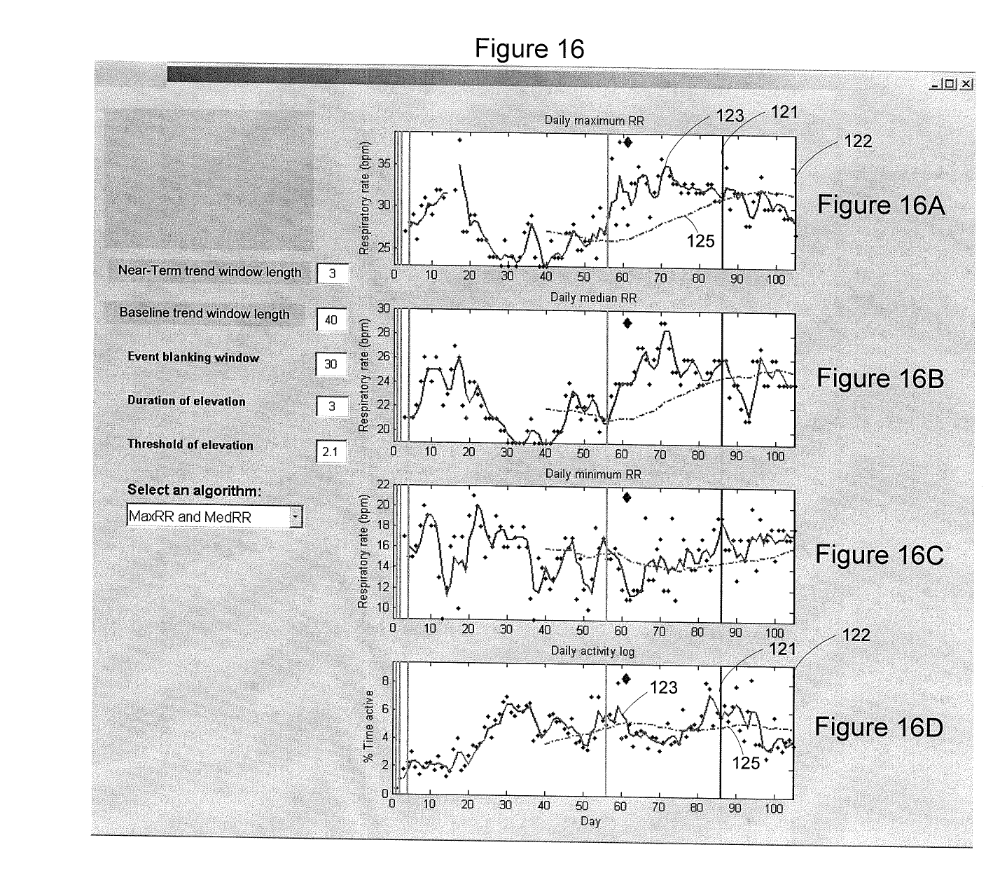

FIG. 16 shows plots of different respiration rate metrics and activity data associated with methodologies for detecting early onset of worsening heart failure in accordance with embodiments of the present invention. FIGS. 16A, 16B, and 16C are plots of near-term averages 123 and baseline averages 125 for MaxRR, MedRR, and MinRR, respectively, developed from respiration data for a particular patient. FIG. 16D is a plot of patient activity data (e.g., accelerometer data). Two heart failure events are shown as lines 121 and 122 for this representative patient.

In the context of FIG. 16, an HF event is declared whenever the patient has signs and/or symptoms consistent with congestive HF and (a) the patient receives unscheduled intravenous therapy (e.g., intravenous (IV) diuretics, IV inotropes, IV vasoactive drugs), oral thiazide, or ultrafiltration therapy that does not involve formal in-patient hospital admission, regardless of the setting (i.e. in an emergency room setting, in the physician's office, etc.) or (b) one of the patient's reasons for admission to the hospital was HF and the patient received an augmented heart failure regimen with oral or intravenous medications or ultrafiltration therapy (formal hospital admission is defined as admission to the hospital that includes a calendar date change).

Arranged vertically along the left panel of FIG. 16 are parameters that are used by the HF event detection algorithm that processes the data of FIGS. 16A-16D. These parameters include the near-term window length (e.g., 3 days), baseline window length (e.g., 40 days), event blanking window (e.g., 30 days), duration of elevation (e.g., 3 days), and threshold of elevation (e.g., 2.1). The short- and baseline windows have been previously discussed. The event blanking window represents a period of time following generation of an alert for which generation (or communication) of a subsequent alert is not permitted. The purpose of the event blanking window is to prevent repeated alerting of the same patient condition that generated an initial alert.

It is noted that the baseline average respiration metrics are updated during the event blanking window. There are conditions, however, when updating of the baseline average respiration metrics may not be permitted or is modified. For example, it may be desirable not to permit updating of the baseline average respiration metrics once it is determined that the patient is in a disease condition. In this case, the baseline average respiration metrics are not updated until the disease condition is resolved. In another scenario, it may be desirable to permit updating of the baseline average respiration metrics but in a modified form. For example, respiration rate data within the baseline window may be weighted in a manner that de-emphasizes data collected during or surrounding an HF event.

The duration of elevation parameter and threshold of elevation operate cooperatively. The threshold of elevation was discussed previously, and, in general terms, modifies the relationship between detection sensitivity and rate of false positives. The duration of elevation represents the amount of time the near-term average respiration rate metric must exceed its associated baseline average respiration rate metric before an alert condition is considered verified.

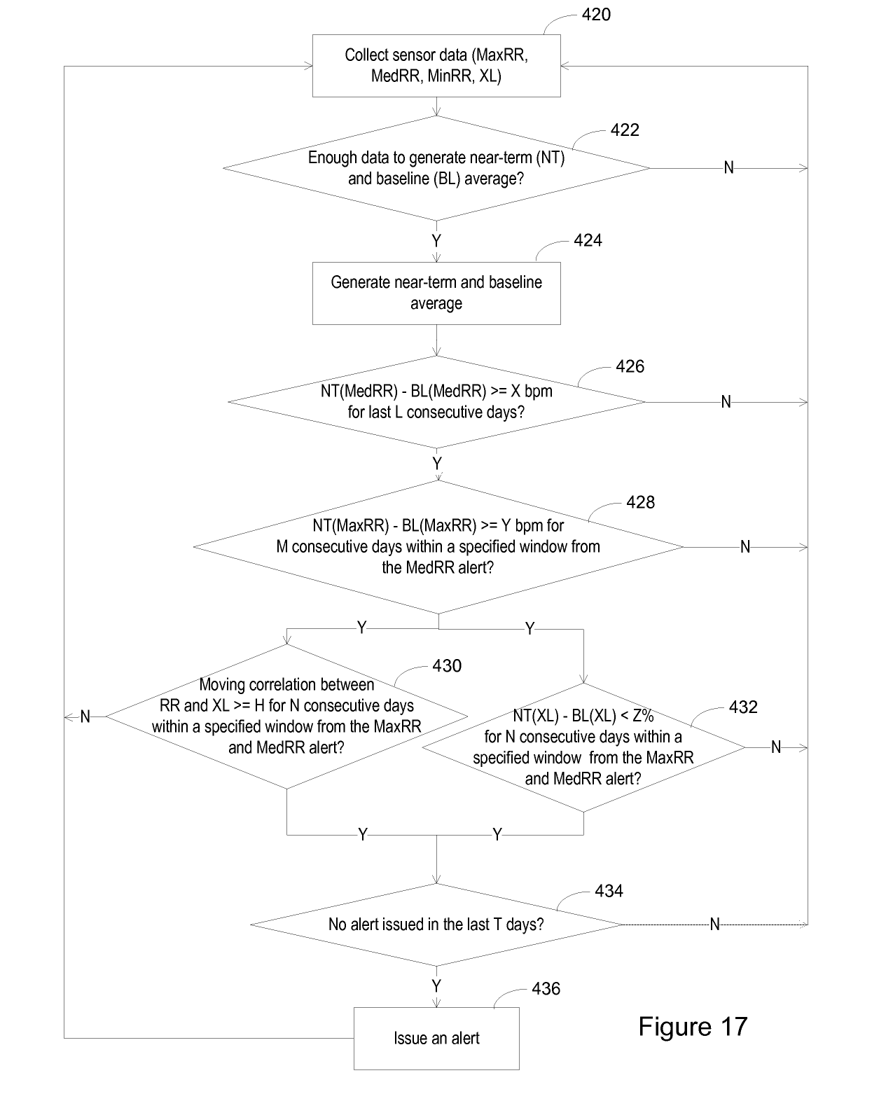

FIG. 17 is a flow chart illustrating various processes for detecting early onset of worsening of a patient's heart failure status in accordance with various embodiments of the invention. According to the embodiment shown in FIG. 17, sensor data is collected 420 for the patient, which includes MaxRR and MedRR, and optionally MinRR and patient activity data, denoted as XL (accelerometer) data. A check 422 is made to determine if a sufficient amount of data has been collected to generated short- and baseline averages for the collected sensor data. If so, short- and baseline averages are generated 424 for the collected sensor data.

At decision block 426, a difference between NT(MedRR) and BL(MedRR) is compared to a threshold, X (bpm). If this difference exceeds the threshold, X, for the last L consecutive days (or x of the last y days), then process flow continues to decision block 428. At decision block 428, a difference between NT(MaxRR) and BL(MaxRR) is compared to a threshold, Y (bpm). If this difference exceeds the threshold, Y, for the last M consecutive days (or x of the last y days) within a specified window from the last alert condition (referred to here as MedRR alert), then process flow continues to optional decision blocks 430 and 432. It is noted that thresholds X and Y may be the same or different, and are preferably optimized for each patient.

Optional decision blocks 430 and 432 implicate processes for determine whether or not the patient has been engaged in activity during the time period of interest. At decision block 430, a moving correlation between respiration rate (RR) and patient activity data (XL) is computed and compared to a threshold, H. If this correlation exceeds the threshold, H, for N consecutive days (or x of the last y days) within a specified window from the MaxRR and MedRR alert condition, then process flow continues to decision block 434.

At decision block 432, a difference between NT(XL)−BL(XL) is computed and compared to a threshold, Z %. If this difference is less than the threshold, Z %, for N consecutive days (or x of the last y days) within a specified window from the MaxRR and MedRR alert condition, then process flow continues to decision block 434. If no alert has been issued in the last T days, as tested at decision block 434, then an alert is issued 436.

It is understood that alert criteria may be used other than, or in addition to, those described above. The following is a representative list of alert criteria that may be used: (a) NT(MinRR)>baseline(MedRR); (b) NT(MedRR>baseline(MaxRR); (c) MaxRR−MedRR>a predetermined threshold; and (d) Variance of respiration rate (Var(RR))>x standard deviation of the baseline.

As previously described, the patient's respiration rate is particularly useful in determining patient status and/or the effectiveness of a prescribed therapy. In one embodiment, the alert is based on the respiration rate. In some scenarios, it is advantageous to employ a multi-sensor approach for more detailed assessment of certain patients or disorders. To this end, respiration and one or more additional physiological signals may be sensed and used together to assess changes in patient status and/or therapy effectiveness. For example, trends of left ventricular (LV) function, heart rate variability, disordered breathing, percent in bi-ventricular pacing, patient activity, weight, heart rate, and/or blood pressure may be useful in determining changes in patient status and therapy effectiveness, particularly for HF patients.

Patient information developed from a patient questionnaire may be used. The patient questionnaire can be presented to the patient via the patient communicator on a periodic basis, such as daily or weekly. In this configuration, the patient communicator is equipped with a user interface, allowing the patient to respond to questions appearing on a display. The patient questionnaire may be programmed to acquire information regarding symptoms that are difficult to acquire automatically such as feelings of fatigue, depression, and/or subjective information related to the patient's health, patient compliance with prescribed therapies, and/or other information useful in the analysis of patient status and therapy effectiveness.

In some embodiments, selecting the alert criteria may involve selecting an algorithm for dynamically changing the alert criteria based on patient status. For example, if the patient's parameter trends generally indicate a decline in patient status, the alert criteria may be automatically modified by the alert module to be more sensitive to changes in patient status. On the other hand, if the patient's physiological parameters generally indicate an improvement in overall health status, the alert criteria may be automatically modified by the alert module to be less sensitive to changes in patient status. This feature automatically reconfigures the alert criteria to avoid overburdening the patient's physician with unnecessary alerts.

In one embodiment, assessment of changes in therapy and/or need for optimization of therapy is based on a single parameter, such as respiration rate. The alert criteria are met when the respiration rate metrics exceed a threshold(s) for a predetermined period of time. When the alert criteria are met, this indicates a decline in therapy effectiveness and the alert signal is generated.

When multiple parameters are tracked, the alert criteria may be based on relationships between the various parameters. For example, if both the respiration rate and LV function are used, then the alert signal may be triggered if both parameters meet or exceed an alert threshold. In an alternative configuration, the alert signal may be triggered if only one parameter meets or exceeds the alert threshold. In yet another configuration, the alert signal may be triggered if one parameter meets or exceeds the alert threshold and the other parameter is trending downward, indicating a worsening patient status.

In certain embodiments, one parameter may be used to automatically alter the alert threshold of another. This technique provides automatic adjustment in the sensitivity of the alert. For example, if the patient's reports of dyspnea or tachypnea indicate this parameter is trending higher, then the threshold for the respiration rate may be adjusted downward so that a lower respiration rate will trigger the alert. This threshold adjustment for the alert criteria allows the alert to be more responsive to the patient's perception of breathlessness, even when the respiration rate may not indicate a change that, when viewed in isolation, would indicate a problem.

In some embodiments the baseline value of the respiration rate may be learned automatically by the device. For example, during an initialization phase, system may make measurements of respiration rate to determine the baseline respiration rate for the patient. The period of time and frequency of measurements used to determine the baseline can be programmable. The alert threshold, in either breaths per minute over the baseline or percentage over the baseline, can be determined input by the physician or automatically determined by the system.

In some embodiments, the alert module may take into account various contextual factors that have an impact on the physiological parameter used to generate the alert. Additional sensors may be used to acquire information which provides a context for detected changes. For example, the patient's respiration rate depends directly on the patient activity. In one scenario, the patient's overall respiration rate may trend upward because he or she has embarked on a new exercise regimen. Without taking the patient's activity level into consideration, an unwarranted alert may be produced. As another example, if the patient is sick, e.g., has pneumonia or other respiratory illness, then the effects of the illness may temporarily cause an increase in the patient's respiration rate. Optimization of therapy may not necessarily be indicated as a response to a temporary illness. Thus, the alert module may take into account the patient's health status in determining whether to generate the alert signal.

An alert signal may be used for various purposes. In one embodiment, the alert signal triggers a communication transmitted to the patient's physician or other health care provider. For example, the communication may involve an email, a telephone message, a fax and/or other type of communication directed to the patient's physician informing the physician of the detected change in therapy effectiveness and/or the need for therapy optimization based. The communication may range from cryptic indication of the change to a multi-level alert that indicates and/or provides an evaluation of the criticality of the change in patient status and/or need for therapy optimization. In some embodiments, the communication may provide additional information about the patient's status. For example, the communication may request that the physician log into the patient management server or the patient's website to view an update on the patient's status.

In some embodiments, the alert signal may trigger an analysis of the patient's therapy. The analysis of patent therapy may make use of information used to generate the alert along with other sensed physiological signals and/or other information. If an analysis of the therapy is performed, the communication to the patient's physician may provide suggestions for modification of the patient's therapy, such as by modifying a prescribed pharmacological therapy and/or by modifying device programming, e.g., re-programmed cardiac pacing parameters. In some implementations, the communication may indicate the need for a change in the type of device the patient is using. For example, the alert module may analyze physiological parameters to determine if a patient needs a device that is capable of providing cardiac resynchronization therapy (CRT) by bi-ventricular and/or biatrial cardiac pacing. If the analysis concludes that CRT is indicated, the communication may include such a recommendation which may require a change in device type.

In some embodiments, the alert signal may trigger an automatic or semi-automatic optimization of therapy. For example, optimization of therapy for HF patients implanted with CRT devices may involve optimizing various parameters of CRT.

CRT, through cardiac pacing, changes the electrical activation sequence of the heart by delivery of pacing pulses to multiple heart chambers. Modification of the electrical activation sequence changes the mechanical contractile sequence of the heart. If effective, the CRT improves the patient's hemodynamic status. CRT parameter optimization may analyze physiological signals and return parameters for CRT optimization based on the analysis of the physiological signals. Parameters for CRT returned by CRT optimization processes may include one or more cardiac pacing parameters such as atrioventricular delay (AVD), interventricular delay (IVD), interatrial delay (IAD), intersite pacing delays, pacing mode, tracking or non-tracking operation, pacing sites, pacing rate limits, and/or other pacing parameters, and/or non-pacing parameters, such as titrating the drugs being taken by the patients. CRT optimization methodologies may reduce the number of CRT recipients who have a less favorable response to CRT, through selecting the most appropriate cardiac pacing parameters.

The physiological signals used for CRT optimization may include cardiac electrical signals including cardiac signals sensed internal to the heart, denoted electrograms (EGMs). From EGMs, the heart's electrical activation sequence can be determined. The EGM may show excessive delays and/or blockages in portions of the heart's electrical conduction system. Exemplary CRT optimization processes based on analysis of cardiac electrical signals are described in commonly owned U.S. Pat. Nos. 7,013,176, 7,113,823, 7,181,285, 7,310,554, and 7,389,141, which are incorporated herein by reference.

Physiological signals used for CRT optimization may include signals associated with the heart's mechanical contractile sequence. In one example, heart sounds, or generally energies resulting from the heart's mechanical vibrations, indicate the mechanical contractile sequence. One particular type of heart sound, known as the third heart sound, or S3, has been found to be associated with heart failure. For example, an increase in S3 activity may indicate elevated filling pressures which may result in the state of decompensated heart failure. S3 amplitude is related to the filling pressure of the left ventricle during diastole. The pitch, or fundamental frequency, of S3 is related to ventricular stiffness and dimension. Chronic changes in S3 amplitude may be correlated to left ventricular chamber stiffness and degree of restrictive filling. An exemplary CRT optimization process based on analysis of heart sounds is described in commonly owned U.S. Patent Application Publication 2004/0127792 and U.S. Pat. No. 7,115,096 which are incorporated herein by reference.

Physiological signals used for CRT optimization may include heart rate from which heart rate variability data may be derived. Heart rate variability (HRV) is the beat-to-beat variability in heart rate. The main component of HRV is respiratory sinus arrhythmia (RSA). Under resting conditions, the healthy individuals exhibit periodic variation in beat to beat intervals with respiration. The heart rate accelerates during expiration and slows during inspiration. Reduction in HRV is a symptom of HF and is related to compromised neurohormonal status. An exemplary CRT optimization process based on analysis of HRV is described in commonly owned U.S. Pat. No. 7,343,199 which is incorporated herein by reference.

Physiological signals used for CRT optimization may include blood pressure signals which are directly related to hemodynamic status. In various examples, blood pressure may be sensed invasively or non-invasively and used to determine CRT parameters. For example, arterial pressure may be measured invasively by placing a pressure catheter in an artery, such as the radial artery. Left ventricular pressure may be measured via a pressure sensor inserted into the left ventricle. Non-invasive measurement of arterial pressure may be performed using a tonometer, phonocardiogram, or other methods. Pressure measurements obtained using these processes, or other processes, may be used to determine CRT parameters. Exemplary CRT optimization processes based on analysis of pressure signals are described in commonly owned U.S. Pat. Nos. 6,666,826, 7,158,830, and 7,409,244 which are incorporated herein by reference.

Other exemplary CRT optimization processes that may be used in conjunction with the methods and systems of the present invention are described in U.S. Pat. No. 7,206,634 which describes therapy optimization based on the use of mechanical sensors, U.S. Pat. No. 7,041,061 which describes therapy optimization based on quantification of wall motion asynchrony using echocardiographic images, U.S. Pat. No. 7,228,174 which describes therapy optimization based on impedance measurements, and U.S. Pat. No. 6,832,113, which describes therapy optimization based on a plethysmogram signal, all of which are incorporated herein by reference.

One or more of the above-referenced CRT optimization processes may be triggered by the alert signal. In some embodiments, the pacing parameters returned from the CRT optimization processes may be automatically implemented to optimize the CRT therapy. Alternatively, the CRT parameters returned from the CRT optimization processes may be presented to the physician as recommended device programming changes. The physician may select the pacing parameters used to optimize CRT. In some embodiments, re-programming the device may be performed remotely by the physician.

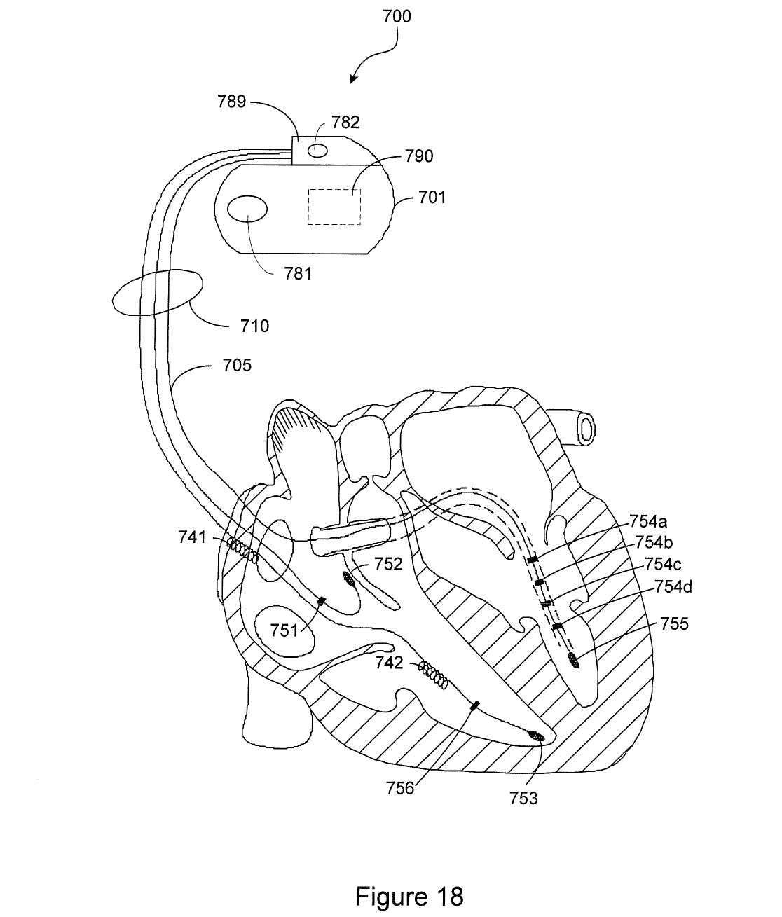

FIG. 18 illustrates a partial view of a patient implantable medical device that may be used to implement processes for detecting early onset of worsening of a patient's heart failure status in accordance with embodiments of the invention. The therapy device 700 illustrated in FIG. 18 may be used to acquire physiological data from which parameter trends may be developed for assessing changes in patient status and/or effectiveness of therapy. The therapy device 700 includes CRM circuitry enclosed within an implantable housing 701. The CRM circuitry is electrically coupled to an intracardiac lead system 710. Although an intracardiac lead system 710 is illustrated in FIG. 18, various other types of lead/electrode systems may additionally or alternatively be deployed. For example, the lead/electrode system may comprise and epicardial lead/electrode system including electrodes outside the heart and/or cardiac vasculature, such as a heart sock, an epicardial patch, and/or a subcutaneous system having electrodes implanted below the skin surface but outside the ribcage.

Portions of the intracardiac lead system 710 are shown inserted into the patient's heart. The lead system 710 includes cardiac pace/sense electrodes 751-756 positioned in, on, or about one or more heart chambers for sensing electrical signals from the patient's heart and/or delivering pacing pulses to the heart. The intracardiac sense/pace electrodes 751-756, such as those illustrated in FIG. 18, may be used to sense and/or pace one or more chambers of the heart, including the left ventricle, the right ventricle, the left atrium and/or the right atrium. The CRM circuitry controls the delivery of electrical stimulation pulses delivered via the electrodes 751-756. The electrical stimulation pulses may be used to ensure that the heart beats at a hemodynamically sufficient rate, may be used to improve the synchrony of the heart beats, may be used to increase the strength of the heart beats, and/or may be used for other therapeutic purposes to support cardiac function consistent with a prescribed therapy.

The lead system 710 includes defibrillation electrodes 741, 742 for delivering defibrillation/cardioversion pulses to the heart. The left ventricular lead 705 incorporates multiple electrodes 754a-754d and 755 positioned at various locations within the coronary venous system proximate the left ventricle. Stimulating the ventricle at multiple locations in the left ventricle or at a single selected location may provide for increased cardiac output in a patients suffering from HF, for example, and/or may provide for other benefits. Electrical stimulation pulses may be delivered via the selected electrodes according to a timing sequence and output configuration that enhances cardiac function. Although FIG. 18 illustrates multiple left ventricle electrodes, in other configurations, multiple electrodes may alternatively or additionally be provided in one or more of the right atrium, left atrium, and right ventricle. Optimization of CRT may involve selecting electrodes used to deliver pacing therapy.

Portions of the housing 701 of the implantable device 700 may optionally serve as one or more multiple can 781 or indifferent 782 electrodes. The housing 701 is illustrated as incorporating a header 789 that may be configured to facilitate removable attachment between one or more leads and the housing 701. The housing 701 of the therapy device 700 may include one or more can electrodes 781. The header 789 of the therapy device 700 may include one or more indifferent electrodes 782. The can 781 and/or indifferent 782 electrodes may be used to deliver pacing and/or defibrillation stimulation to the heart and/or for sensing electrical cardiac signals of the heart.

The cardiac electrodes can be used in conjunction with appropriate circuitry 790 disposed within the housing 701 of the therapy device 700 to sense transthoracic impedance and to develop a respiration signal from the transthoracic impedance measurements. As previously discussed, various respiration parameters can be determined from the respiration signal and a trend of the respiration parameter developed, although these processes may or may not be implemented by the therapy device 700. The respiration parameter is used to assess changes in therapy effectiveness or patient status.

In some embodiments, the therapy device 700 may also include sensors and/or circuitry for determining additional physiological parameters that may be useful in assessing therapy effectiveness. For example, the therapy device 700 may include an accelerometer used for sensing patient activity, may include circuitry for determining heart rate variability from the electrogram signal, may include circuitry to detect disordered breathing episodes, and/or may include circuitry for sensing various other parameters.

Communications circuitry is disposed within the housing 701 for facilitating communication between the CRM circuitry and a patient-external device, such as an external programmer or patient communicator coupled to a patient management server. In some embodiments the therapy device may include a sensor configured to sense the metabolic need so that the pacing rate can be adapted to accommodate the patient's metabolic need.

In certain embodiments, the therapy device 700 may include circuitry for detecting and treating cardiac tachyarrhythmia via defibrillation therapy and/or anti-tachyarrhythmia pacing (ATP). Configurations providing defibrillation capability may make use of defibrillation coils 741, 742 for delivering high energy pulses to the heart to terminate or mitigate tachyarrhythmia.

CRM devices using multiple electrodes, such as illustrated herein, are capable of delivering pacing pulses to multiple sites of the atria and/or ventricles during a cardiac cycle. Certain patients may benefit from activation of parts of a heart chamber, such as a ventricle, at different times in order to distribute the pumping load and/or depolarization sequence to different areas of the ventricle. A multi-electrode pacemaker has the capability of switching the output of pacing pulses between selected electrode combinations within a heart chamber during different cardiac cycles.

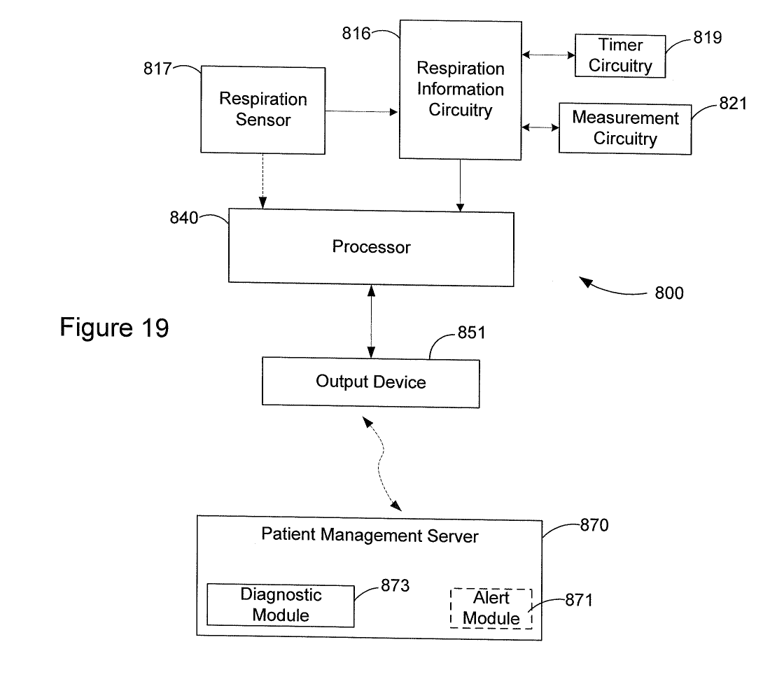

FIG. 19 is a block diagram of a medical system 800 that may implement various diagnostic, alert, and/or therapy processes in accordance with various embodiments. In general terms, the system 800 shown in FIG. 19 is particularly well suited for assessing a patient's heart failure status and, more particularly, for detecting early onset of worsening of a patient's heart failure condition and generating an alert regarding same. The system 800 is preferably configured to implement respiration rate trending techniques are described herein for detecting a patient's early onset of worsening heart failure.

According to various embodiments, the medical system 800 includes a respiration sensor 817 configured to generate a signal indicative of patient respiration. As previously discussed, the respiration sensor 817 may be an implantable sensor or an external sensor (or a sensor that combines implantable and external components). The system 800 includes respiration information circuitry 816 coupled to the respiration sensor 817 and configured to make various respiration measurements using the signal indicative of patient respiration provided by the respiration sensor 817. The respiration information circuitry 816 is preferably configured to measure a median respiration rate (MedRR) and a maximum respiration rate (MaxRR), and may optionally be configured to measure a minimum respiration rate (MinRR).

FIG. 19 shows timer circuitry 819 and measurement circuitry 821 respectively coupled to respiration information circuitry 816. Timer circuitry 819 is configured to time a plurality apertures in a manner discussed hereinabove. The measurement circuitry 821 is configured to measure a respiration rate for each breath cycle of each aperture, estimate a respiration rate for each aperture based on the measured respiration rates, and determine MedRR and MaxRR from the estimated respiration rates in a manner previously described above.

The processor 840 is coupled to the respiration information circuitry 816 and configured to determine whether an abnormality exists in MedRR and whether an abnormality exists in MaxRR, preferably in a manner previously described. The processor 840 may further be configured to determine whether an abnormality exists in MinRR. An output device 851 is coupled to the processor 840 and configured to generate an output indicative of the patient's tachypnea status in response to determining the abnormality in MedRR and MaxRR.

The processor 840 may be configured to determine whether there is an abnormality in MinRR, and the output device 851 may be configured to generate an output indicative of the patient's heart failure status based on the abnormality determination in MedRR, MaxRR, and MinRR. The processor 840 may be configured to determine the patient's heart failure status by determining whether abnormality (e.g., elevation) in MedRR, MaxRR, and MinRR are within a predetermined range of abnormality, and may further cooperate with the output device 851 to generate an output indicating that the patient's heart failure status may be worsening.

The processor 840 and the output device 851 may be implemented to cooperatively generate an output indicative of the patient's heart failure status based at least in part on the processor 840 determining whether a near-term measure of MinRR is greater than a baseline for MedRR. The processor 840 and the output device 851 may be implemented to cooperatively generate an output indicative of the patient's heart failure status based at least in part on the processor 840 determining whether a near-term measure of MedRR is greater than a baseline for MaxRR.

The output device 851 may be configured to produce an alert in response to conditions described above, and communications circuitry of the system may be configured to communicate the alert to a networked patient management system 870 or other receiving device or system (e.g., communicator or PC). The patient management system 870 may include an alert module 871 and a diagnostic module 873 for implementing a respiration rate trending and alert algorithm in accordance with various described embodiments of the present invention. As previously discussed, functions performed by the alert module 871 and/or the diagnostic module 873 may alternatively be implemented by the processor 840 or other component(s) of the medical system 800.

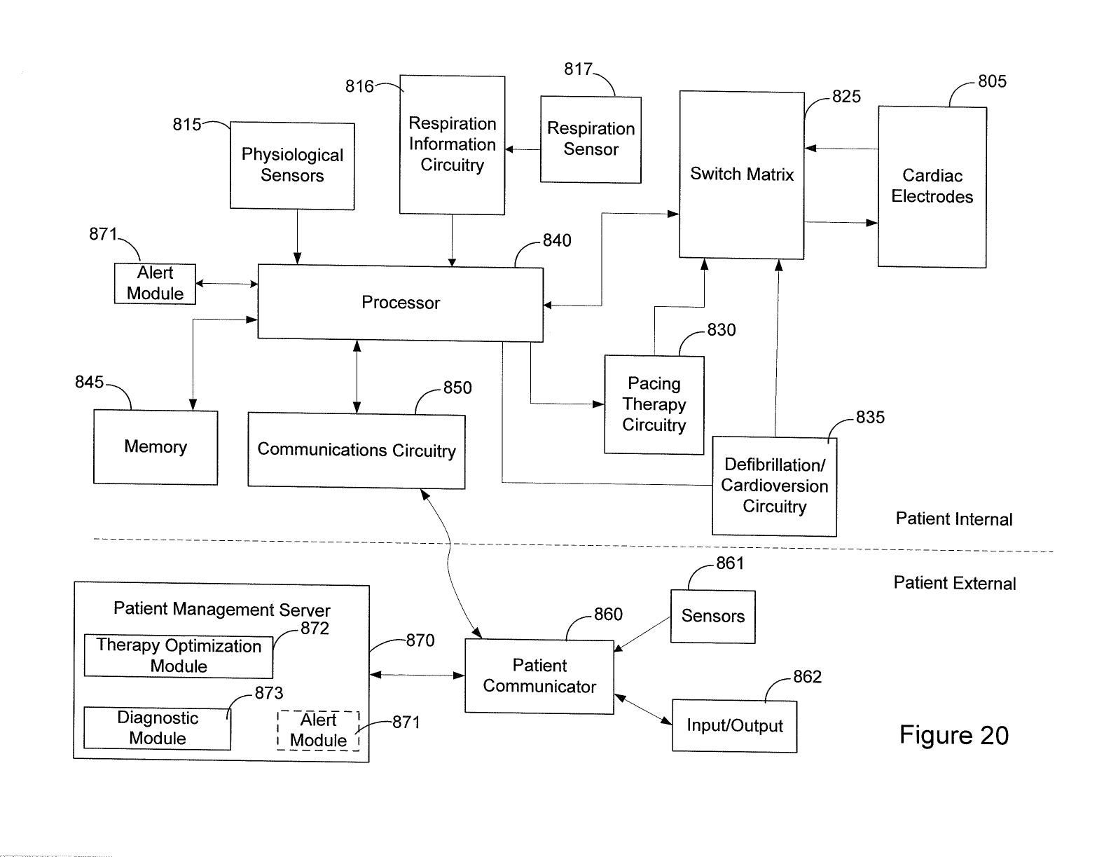

FIG. 20 is a block diagram of one embodiment of a medical system 800 that may be configured to implement respiration rate trending and alert processes in accordance with various embodiments of the present invention. The system 800 includes a patient internal device (implantable CRM device) that incorporates pacing therapy circuitry 830 configured to deliver pacing pulses to a heart via cardiac electrodes 805. The implantable CRM device may optionally include defibrillation/cardioversion circuitry 835 configured to deliver high energy defibrillation or cardioversion stimulation to the atria or ventricles of the heart for terminating tachyarrhythmias.

The electrodes 805 are coupled to switch matrix 825 circuitry used to selectively couple electrodes 805 to other components of the CRM device. The electrodes 805 may be used in conjunction with respiration sensor 816 (e.g., transthoracic impedance circuitry) to sense the patient's respiration signal. Additional physiological sensors 815 may also be included in the CRM device.

The processor 840 controls the therapy and sensing operations of the CRM device. Additionally, the processor 840 manages data storage operations to allow storage in memory 845 signals, parameter measurements and/or parameter trends developed using the respiration sensor data and, if used, the data from the other physiological sensors 815. The processor 840 preferably implements respiration rate trending and alert logic as described herein for determining a patient's tachypnea and/or heart failure status, such as early onset of worsening of a patient's heart failure. In some automatic configurations, the CRM device may include the alert module that assesses changes in therapy effectiveness and generates the alert signal based on these changes. Additionally or alternatively, the CRM device may include diagnostic or therapy modification circuitry. The diagnostic circuitry may assess the parameter trends stored in memory to diagnose a disease or to assess the progression of a disease or symptoms associated with the disease. Responsive to a signal generated by the alert module, the processor 840 may automatically initiate a therapy optimization procedure.

A CRM device typically includes a battery power supply (not shown) and communications circuitry 850 for communicating with the external patient communicator 860, device programmer (not shown) or other patient-external device. Data stored in the memory of the CRM device, such as signals, measurements or trends from the respiration signal and/or other physiological sensor signals, can be transferred from the memory 845 of the CRM device to the patient communicator 860 via the communications circuitry 850. Transfer of this information may be performed periodically, on demand, or in response to a triggering event.

In some embodiments, the patient communicator 860 receives the information from the CRM device and forwards it to the patient management server 870 for assessment of changes in a patient's tachypnea and/or heart failure status and/or therapy effectiveness. The patient management 870 server may optionally include an alert module 871, configured to analyze the information received from the CRM device via the patient communicator. The alert module is configured to generate alert signals based on comparison of parameters to alert criteria. The patient management server 870 may optionally include a diagnostic module 873 for diagnosing a disease presence and/or monitoring the progression, regression, or status quo of a disease condition. The patient management server 870 may optionally include a therapy optimization module 872 configured to evaluate the patient's condition and assess therapy settings based on the parameter information received from the CRM device. After analysis, modification of therapy parameters may be transferred to the CRM device to automatically effect changes in the patient's therapy in some implementations.

In some embodiments, the patient communicator 860 may also include circuitry and/or software to make parameter measurements and develop parameter trends. The alert module, therapy optimization module, and/or diagnostic module may be fully or partially disposed in the patient communicator 860 imbuing the patient communicator 860 with partial or full functionality to analyze the parameter values, develop parameter trends, assess changes in patient status and/or therapy effectiveness, and determine appropriate therapy adjustments. In this configuration, the patient communicator 860 may make recommendations for therapy optimization and/or download optimized therapy parameters to the CRM device, and/or trigger the CRM device to implement processes for determining optimized parameters.

The patient communicator 860 may be coupled to various sensors 861 for acquiring information about patient parameters, e.g., patient externally acquired parameters, in addition to the implantably acquired respiration parameters. In certain embodiments, the sensors 861 may include a blood pressure sensor and weight scale. The sensors 861 and patient communicator 860 may employ wireless communication technology such as Blue Tooth, or other RF telemetry protocols. The patient may access the sensors 861 in accordance with a prescribed testing schedule. For example, the patient may measure his or her weight and blood pressure at periodic intervals and this information may be communicated from the sensors 861 to the patient communicator 860.

The patient communicator 860 may be coupled to an input/output device 862 including a keyboard, pointing device, touch panel or other input device, and a display. The patient may interact the input/output device to answer questionnaires displayed to the patient on the display. The patient's answers to the questions may be trended along with the measurements acquired from the sensors 815, 816 coupled to the CRM device or sensors 861 coupled to the patient communicator 860. The additional parameters may be used along with the respiration parameters to assess changes in therapy effectiveness.

The components, functionality, and structural configurations depicted herein are intended to provide an understanding of various features and combination of features that may be incorporated in an implantable or patient-external medical device or system. For example, an external respiration sensor may be used to acquire patient respiration information, and an external processor or other logic device may be employed to compute MedRR, MaxRR, and optionally MinRR metrics, determine abnormality in any of the MedRR, MaxRR, and MinRR metrics, and generate an output indicative of the patient's tachypnea status and/or heart failure status based on the abnormality determination in MedRR, MaxRR, and MinRR. It is understood that a wide variety of such device or system configurations are contemplated, ranging from relatively sophisticated to relatively simple designs. As such, particular implantable/external or cardiac monitoring and/or stimulation device configurations may include particular features as described herein, while other such device configurations may exclude particular features described herein.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A method, comprising:
sensing respiration and measuring a median respiration rate (MedRR) and a maximum respiration rate (MaxRR) using the sensed respiration;
determining whether an abnormality exists in MedRR;
determining whether an abnormality exists in MaxRR; and
generating an output indicative of the patient's tachypnea status in response to determining the abnormality in MedRR and MaxRR.

2. The method of claim 1, wherein determining the abnormality in MedRR and MaxRR comprises:
computing a baseline for MedRR and MaxRR, respectively;
computing a near-term measure for MedRR and MaxRR, respectively; and
determining the abnormality in MedRR and MaxRR based on a comparison of a difference between the baseline and near-term measure relative to a predetermined threshold for MedRR and MaxRR, respectively.

3. The method of claim 2, wherein:
the baseline comprises one of a mean of a first predetermined period, a median of the first predetermined period, a weighted average of the first predetermined period, a predetermined value based on a population, and a predetermined value based on a patient's history; and
the near-term measure comprises one of a mean of a second predetermined period and a median of the second predetermined period.

4. The method of claim 3, wherein the first predetermined period is longer than the second predetermined period or overlaps with at least a portion of the second pre-determined period.

5. The method of claim 2, wherein determining the abnormality in MedRR and MaxRR comprises one of:
determining whether the difference exceeds the predetermined threshold respectively for MedRR and MaxRR for a predetermined duration of time; and
determining whether the difference exceeds the predetermined threshold respectively for MedRR and MaxRR for a predefined percentage of the predetermined duration of time.

6. The method of claim 2, wherein determining the abnormality in MedRR and MaxRR comprises determining whether the respective abnormality in MedRR and MaxRR occur within a predetermined time window.

7. The method of claim 2, wherein:
determining the abnormality in MedRR and MaxRR comprises at least one of:
determining whether the difference exceeds the predetermined threshold respectively for MedRR and MaxRR for a predetermined duration of time;
determining whether the difference exceeds the predetermined threshold respectively for MedRR and MaxRR for a predefined percentage of the predetermined duration of time; and
determining whether the respective abnormality in MedRR and MaxRR occur within a predetermined time window; and
the method comprising adjusting at least one of the predetermined threshold, predetermined duration, predefined percentage, and predetermined time window based on desired performance requirements.

8. The method of claim 2, comprising adjusting the respective predetermined threshold based on the respective baseline.

9. The method of claim 1, comprising determining whether the patient is engaged in patient activity, wherein generating the output comprises generating an output indicating that the patient's tachypnea status is not caused by patient activity in response to determining the abnormality in MedRR and MaxRR and determining that the patient is not engaged in patient activity.

10. The method of claim 1, comprising:
measuring a minimum respiration rate (MinRR) using the sensed respiration;
determining whether there is an abnormal elevation in MinRR; and
generating an output indicative of the patient's tachypnea status in response to determining the abnormality in MedRR and MaxRR and absence of abnormal elevation in MinRR.

11. The method of claim 1, wherein determining the abnormality in MedRR and MaxRR comprises:
computing a variation of a baseline for MedRR and MaxRR, respectively;
computing a variation of a near-term measure for MedRR and MaxRR, respectively; and
determining the abnormality in MedRR and MaxRR based on a comparison of a difference between the baseline and near-term measure variation relative to a predetermined threshold for MedRR and MaxRR, respectively.

12. The method of claim 1, comprising:
measuring a minimum respiration rate (MinRR) using the sensed respiration;
determining whether there is an abnormality in MinRR; and
generating an output indicative of the patient's heart failure status based on the abnormality determination in MedRR, MaxRR, and MinRR.

13. The method of claim 12, wherein generating the output indicative of the patient's heart failure status comprises generating the output indicative of the patient's heart failure status based at least in part on determining whether a near-term measure of MinRR is greater than a baseline for MedRR.

14. The method of claim 12, wherein generating the output indicative of the patient's heart failure status comprises generating the output indicative of the patient's heart failure status based at least in part on determining whether a near-term measure of MedRR is greater than a baseline for MaxRR.

15. The method of claim 1, wherein the output comprises an alert, and the method comprises communicating the alert to an external system.

16. A medical system, comprising:
a respiration sensor configured to generate a signal indicative of patient respiration;
respiration information circuitry coupled to the respiration sensor and configured to measure a median respiration rate (MedRR) and a maximum respiration rate (MaxRR) using the signal indicative of patient respiration;
a processor coupled to the respiration information circuitry and configured to determine whether an abnormality exists in MedRR and whether an abnormality exists in MaxRR; and
an output device coupled to the processor and configured to generate an output indicative of the patient's tachypnea status in response to determining the abnormality in MedRR and MaxRR.

17. The system of claim 16, wherein the respiration information circuitry comprises:
timer circuitry configured to time a plurality apertures; and
measurement circuitry configured to measure a respiration rate for each breath cycle of each aperture, estimate a respiration rate for each aperture based on the measured respiration rates, and determine MedRR and MaxRR from the estimated respiration rates.

18. The system of claim 16, wherein the processor is configured to compute a baseline for MedRR and MaxRR, respectively, compute a near-term measure for MedRR and MaxRR, respectively, and determine the abnormality in MedRR and MaxRR based on a comparison of a difference between the baseline and near-term measure relative to a predetermined threshold for MedRR and MaxRR, respectively.

19. The system of claim 18, wherein:
the baseline comprises one of a mean of a first predetermined period, a median of the first predetermined period, a weighted average of the first predetermined period, a predetermined value based on a population, and a predetermined value based on a patient's history; and
the near-term measure comprises one of a mean of a second predetermined period and a median of the second predetermined period.

20. The system of claim 19, wherein the first predetermined period is longer than the second predetermined period or overlaps with at least a portion of the second pre-determined period.

21. The system of claim 18, wherein the processor is configured to determine the abnormality in MedRR and MaxRR by at least one of:
determining whether the difference exceeds the predetermined threshold respectively for MedRR and MaxRR for a predetermined duration of time; and
determining whether the difference exceeds the predetermined threshold respectively for MedRR and MaxRR for a predefined percentage of the predetermined duration of time.

22. The system of claim 18, wherein the processor is configured to determine whether the respective abnormality in MedRR and MaxRR occur within a predetermined time window.

23. The system of claim 18, wherein the processor is configured to determine the abnormality in MedRR and MaxRR by at least one of:
determining whether the difference exceeds the predetermined threshold respectively for MedRR and MaxRR for a predetermined duration of time;
determining whether the difference exceeds the predetermined threshold respectively for MedRR and MaxRR for a predefined percentage of the predetermined duration of time; and
determining whether the respective abnormality in MedRR and MaxRR occur within a predetermined time window;
wherein the processor is configured to adjust at least one of the predetermined threshold, predetermined duration, predefined percentage, and predetermined time window based on desired performance requirements.

24. The system of claim 18, wherein the processor is configured to adjust the respective predetermined threshold based on the respective baseline.

25. The system of claim 16, wherein the processor is configured to determine whether the patient is engaged in patient activity, and the output device is configured to generate an output indicating that the patient's tachypnea status is not caused by patient activity in response to determining the abnormality in MedRR and MaxRR and determining that the patient is not engaged in patient activity.

26. The system of claim 16, wherein:
the respiration information circuitry is configured to measure a minimum respiration rate (MinRR) using the signal indicative of patient respiration;
the processor is configured to determine whether there is an abnormal elevation in MinRR; and
the output device is configured to generate an output indicative of the patient's tachypnea status in response to determining the abnormality in MedRR and MaxRR and absence of abnormal elevation in MinRR.

27. The system of claim 16, wherein the processor is configured to determine the abnormality in MedRR and MaxRR by:
computing a variation of a baseline for MedRR and MaxRR, respectively;
computing a variation of a near-term measure for MedRR and MaxRR, respectively; and
the processor is configured to determine the abnormality in MedRR and MaxRR based on a comparison of a difference between the baseline and near-term measure variation relative to a predetermined threshold for MedRR and MaxRR, respectively.

28. The system of claim 16, wherein:
the respiration information circuitry is configured to measure a minimum respiration rate (MinRR) using the signal indicative of patient respiration;
the processor is configured to determine whether there is an abnormality in MinRR; and
the output device is configured to generate an output indicative of the patient's heart failure status based on the abnormality determination in MedRR, MaxRR, and MinRR.

29. The system of claim 28, wherein the processor and the output device cooperate to generate the output indicative of the patient's heart failure status based at least in part on determining whether a near-term measure of MinRR is greater than a baseline for MedRR.

30. The system of claim 28, wherein the processor and the output device cooperate to generate the output indicative of the patient's heart failure status based at least in part on determining whether a near-term measure of MedRR is greater than a baseline for MaxRR.

31. The system of claim 16, wherein the output device is configured to produce an alert and the system comprises communications circuitry configured to communicate the alert to a networked patient management system.

32. A medical system, comprising:
means for sensing respiration and measuring a median respiration rate (MedRR) and a maximum respiration rate (MaxRR) using the sensed respiration;
means for determining whether an abnormality exists in MedRR;
means for determining whether an abnormality exists in MaxRR; and
means for generating an output indicative of the patient's tachypnea status in response to determining the abnormality in MedRR and MaxRR.
